(19) United States
(12) Patent Application Publication
(10) Pub. No.: US 2026/0134760 A1
(43) Pub. Date: May 14, 2026

(54) METHODS, SYSTEMS, AND APPARATUSES FOR A GAME INFLUENCED BY SPECTATOR ACTIVITY

(71) Applicant: Leon I. Rosenberg, Cherry Hill, NJ (US)

(72) Inventor: Leon I. Rosenberg, Cherry Hill, NJ (US)

(21) Appl. No.: 19/120,122

(22) PCT Filed: Oct. 12, 2023
(86) PCT No.: PCT/US2023/076736

§ 371 Date: Apr. 10, 2025

Related U.S. Application Data
(60) Provisional application No. 63/415,557, filed on Oct. 12, 2022.
(60) Provisional application No. 63/424,037, filed on Nov. 9, 2022.
(60) Provisional application No. 63/425,948, filed on Nov. 16, 2022.
(60) Provisional application No. 63/507,002, filed on Jun. 8, 2023.
(60) Provisional application No. 63/518,668, filed on Aug. 10, 2023.

(51) Int. Cl. G07F 17/32; A63F 13/212; A63F 13/86

(52) U.S. Cl. CPC G07F 17/3288; A63F 13/212; A63F 13/86

(57) ABSTRACT

A system for game play influenced by spectator activity comprising a data communication device for transmitting and/or receiving electronic and/or electromagnetic signals of spectator data, and a memory having computer instructions executable by a processor. The memory is configured to execute a spectatable event having a variable game parameter, receive spectator data from a participant spectator of the spectatable event via the data communication device, wherein the spectator data is based on a physical activity and/or any activity of the participant spectator, and update the variable game parameter of the spectatable event based on the received spectator data in real time.

### TECHNICAL FIELD

The present disclosure generally relates to a method, system, and an apparatus for gaming, exercise and entertainment. In particular, the present disclosure relates to methods, systems, and apparatuses for providing and playing a game influenced by spectator activity, and for promoting physical activity, social connection and community connection of an individual participating in a spectatable event.

U.S. patent application Nos. 63/415,557; 63/424,037; 63/425,948; 63/507,002; 63/518,668 are hereby incorporated by reference in their entirety for all purposes.

### BACKGROUND

There is a health epidemic of physical inactivity; physical inactivity is the fourth leading cause of global mortality according to the World Health Organization (2010). There is also a health epidemic of social isolation and loneliness. The 2023 U.S. Surgeon General's advisory report notes that loneliness and social isolation increase the risk of premature death by 26% and 29% respectively. Social isolation is defined as having few social relationships, social roles, group memberships, and infrequent social interaction. According to the Surgeon General's report, approximately half of the U.S. adults report experiencing loneliness, with some of the highest rates among young adults. One conclusion is that since loneliness and social isolation together affect nearly 50% of individuals, loneliness and social isolation are more widespread than many other health issues of our day including smoking (12.5% of U.S. adults), diabetes (14.7%), and obesity (41.9%). The Surgeon General advisory report also references data that shows that social isolation is worse than all of the following: smoking 15 cigarettes per day; drinking 6 alcoholic drinks daily; physical inactivity; obesity; and air pollution. Social isolation is an independent risk factor for deaths from all causes, including deaths caused by diseases.

### BRIEF SUMMARY

Embodiments of the present disclosure are directed to playing a game involving player combatants and participant spectators either in the stands, in remote locations, and/or elsewhere. In exemplary embodiments, the subject disclosure provides a system for game play influenced by spectator activity comprising: a data communication device for transmitting and/or receiving electronic and/or electromagnetic signals of spectator data; and a memory having computer instructions executable by a processor configured to: execute a spectatable event having a variable game parameter, receive spectator data from a participant spectator of the spectatable event via the data communication device, wherein the spectator data is based on a physical activity of the participant spectator, and update the variable game parameter of the spectatable event based on the received spectator data in real time.

The system can further include a second data communication device that is remote from the first data communication device of the system and configured for transmitting spectator data of a participant spectator at a remote location. The second data communication device, herein referred to as a spectator device for generating the spectator data and/or transmitting the spectator data to the data communication device, can be a smart device, e.g., a smart phone, smart watch, a wearable, the Wearable System and/or the like, or any other device. The spectator device can be exercise equipment and/or noise maker and/or energy producing and/or capturing device. The processor is configured to update the variable game parameter upon reaching a predetermined threshold value. The processor is further configured to output to the participant spectator a winner of the spectatable event and spectator data analytics. The spectator data is a quantifiable measure of the actions and/or activities of the participant spectator.

The subject disclosure also provides a method of promoting physical activity of an individual of a spectatable event (also referred to herein as a participant spectator) comprising: providing a spectatable event having a variable game parameter; providing a data communication device for transmitting and/or receiving electronic and/or electromagnetic signals of spectator data; providing a means for receiving spectator data from a participant spectator of the spectatable event via the data communication device, wherein the spectator data is based on a physical exercise activity of the participant spectator; and updating the variable game parameter of the spectatable event based on the spectator data from the participant spectator.

The subject disclosure also provides an apparatus comprising a memory having computer instructions executable by a processor configured to: receive spectator data from a participant spectator of a spectatable event based on a variable parameter, wherein the spectator data is based on a physical exercise activity of the participant spectator; and update the variable parameter of the spectatable event based on the received spectator data in real time.

### BRIEF DESCRIPTION OF ACCOMPANYING DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings, exemplary embodiments. It should be understood, however, that the subject disclosure is not limited to the precise arrangements and instrumentalities shown.

FIG. 1 illustrates an exemplary block diagram of a system according to an exemplary embodiment of the present disclosure; and

FIG. 2 illustrates an exemplary block diagram of a computer system according to an exemplary embodiment of the present disclosure.

### DETAILED DESCRIPTION

Reference will now be made in detail to the various exemplary embodiments of the subject disclosure and illustrated in the accompanying drawings. Wherever possible, the same or like reference numbers will be used throughout the drawings to refer to the same or like features. It should be noted that the drawings are in simplified form and are not drawn to a precise scale.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the invention is by way of example, and the scope of the disclosure is not limited to the exact details shown or described. An exemplary embodiment of the subject disclosure is described in the context of a game played with a ball and two goals; however, the subject disclosure may be applied to any sporting event, or any other type of real-life competitive event where spectator involvement can be and/or is desirable. Various features and sub-combinations of the subject disclosure may be employed without reference to other features and sub-combinations.

It should also be appreciated that the exemplary embodiment is only an example and is not intended to limit the scope or applicability of the disclosure in any way. Rather, the foregoing detailed description will provide those skilled in the art with a convenient road map for implementing various embodiments. Additionally, the foregoing disclosure encompasses multiple distinct examples and embodiments with independent utility. While these examples and embodiments have been disclosed in a particular form, the specific examples and embodiments disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently.

Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the foregoing are examples of the disclosure, additional examples are also possible. Thus, the following description should not be taken as limiting the scope of the disclosure which is defined by the appended claims along with their full scope of equivalents. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or Dis an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

Studies show that being a spectator (e.g., sports fan) can be a physical experience. However, during, before and after most games, the role of spectators is passive as they do not have any direct influence on the decision-making process, or outcome of the game, but spectator behavior is one in-direct external factor that can influence an athlete's performance during a game due to evaluative attitude, encouragement, discouragement, or other emotion, and/or sentiment being conveyed. Just as sports and games have changed over the millennia, the subject matter of the following description provides subject matter that enables spectators and other entities actively contributing, influencing and impacting the game while it is occurring through their production of game energy.

The subject disclosure provides a system and apparatus for game play influenced by spectator activity comprising: a data communication device for transmitting and/or receiving electronic and/or electromagnetic signals of spectator data; and a memory having computer instructions executable by a processor configured to: generate data corresponding to a spectatable event having a variable game parameter, receive spectator data from a participant spectator of the spectatable event via the data communication device, wherein the spectator data is based on a physical activity and/or other activity of the participant spectator, and update the variable game parameter of the spectatable event based on the received spectator data in real time.

The subject disclosure provides a method of promoting physical exercise activity of a participant spectator comprises: providing a spectatable event having a variable game parameter(s); providing a data communication device for transmitting and/or receiving electronic and/or electromagnetic signals of spectator data; receiving spectator data from a participant spectator of the spectatable event via the data communication device, wherein the spectator data is based on a physical exercise activity of the participant spectator; and updating the variable game parameter of the spectatable event based on the spectator data from the participant spectator.

In exemplary embodiments, a method or apparatus includes providing and playing a game and/or competition where spectators, in person (e.g., in the stands of a stadium) and/or in remote locations anywhere in the world can participate, influence, impact and alter the outcome of the game as a result of the energy that is generated by the participant spectators physically exercising, moving, playing online games, cheering verbally, generating noise with noisemakers, and/or other actions related to the game, while player combatants are playing the game and/or at other times. The energy is generated using energy-generating and energy collecting devices, cheering, noise-making, playing musical instruments, playing sports, exercising and competing and/or playing any type of competition, and/or by playing computer/video games. The energy is then stored, recorded, transferred/transmitted, and received via energy storing, recording, transferring, transmitting, and receiving devices to manipulate the outcome of the game.

In other exemplary embodiments, the systems and methods utilize the involvement and contribution of cheerleaders and coaching staff in energy generation and use. Similar to how cheerleaders encourage in person spectators to perform a physical activity (e.g., make noise) and/or generate energy, the systems and methods allow some cheerleaders to encourage remote spectators to perform a physical activity and/or generate energy, for example, by distributing various content configured to entice those remote spectators into making noise and/or completing an exercise routine. In some embodiments, the systems and methods may be utilized to communicate personalized content configured to entice a specific spectator to perform a physical activity and/or generate energy. The systems and methods described herein may be configured to automate the distribution of personalized content without cheerleader involvement.

The spectatable event includes two teams each having at least one player playing a game to win. The variable game parameter includes at least one value establishing the rules of the game, for example the position and size of the strike zone, number of outs per inning, number of strikes required to strike out, the number of balls to walk, number of innings per game, game etiquette, a position of a goal, a change in an obstacle of the game, a change in game field condition, a movement of an object of the game, a game plan, an offensive game plan, a defensive game plan and/or the like. The variable game parameter is updated upon reaching a predetermined threshold value of generated energy and/or the like. The physical exercise activity may involve an aerobic exercise and/or a muscular strengthening exercise and/or talking, cheering, noisemaking, chanting, singing and/or music making, and/or the like. The data communication device is configured to receive spectator data from the spectator device, which may be a smart phone, smart watch and/or an exercise equipment device and/or the like. The method further comprises outputting to the participant spectator of a winner of a portion of the spectatable event or of the completed spectatable event, and spectator data analytics.

Accordingly, exemplary embodiments of the present disclosure provide the experience of turning spectators into active participants in the game, i.e., participant spectators, e.g., spectators that are viewing and/or participating in the game for purposes of influencing the outcome of the game. Being a participant spectator combines the thinking, efforts, memory, psychology, physical efforts, emotional efforts, energy, skill, strength, ability, communication, cheering, knowledge, teamwork, and/or other characteristics of a direct combatant player of the game and the thinking, memory, psychology, physical efforts, emotional efforts, cognitive efforts, energy, skill, strength, ability, communication, cheering, knowledge, teamwork, and other characteristics of a spectator of the game, which can result in the combatant players feeling closer to the participant spectators, and/or can result in the participant spectators feeling closer to the combatant players and/or can result in the participant spectators feeling closer to each other. It combines the thinking, memory, psychology, physical efforts, emotional efforts, energy, skill, strength, ability, communication, cheering, knowledge, teamwork, and other characteristics of any entities associated with the game, for example, spectators, cheerleaders, player combatants, judges, coaches, and/or the like. Exemplary embodiments of the present disclosure provide the experience of making it more likely that participant spectators will exercise and/or produce game energy while observing games. Exemplary embodiments of the present disclosure provide the experience of making it more likely that player combatants will perform better as a result of the activity of the participant. Exemplary embodiments of the present disclosure provide the experience of making it more likely that participant spectators cheerleaders, player combatants, judges, coaches, and/or the like will all contribute to participant spectators feeling and/or themselves feeling social connection and/or a sense of community.

In another embodiment, the methods and systems allow multiple participant spectators in the game venue (e.g., stadium, arena, field, etc.) and/or in remote or virtual locations to actively participate, influence, impact, affect and/or alter the outcome of the game by generating energy e.g., through exercise and/or other physical and/or mental activity. As such, the game provides for and/or promotes the physical and mental health and conditioning of the participant spectators and/or any entities viewing and/or participating in the game.

In another embodiment, the methods and systems involve participant spectators, players, cheerleaders, coaching staff, judges, and/or any other entity associated with the game in energy generation and storing, recording, transferring, transmitting, and receiving of game play energy to affect the outcome of the game.

In another embodiment, the methods and systems improve energy generation by promoting the production of noise, singing, chanting, music, social connection, a sense of community and/or physical, emotional, and/or cognitive processes by supportive and cheering fans, participant spectators, and/or any other entity viewing and/or participating in the game on the field of play, in the stands and/or in remote locations. In another embodiment, the methods and systems promote the supportive and cheering fans, participant spectators, and/or any other entity viewing or participating in the game on the field of play, in the stands and/or in remote locations to partake in influencing the outcome of the game and/or generate energy. In another embodiment, the methods and systems promote the supportive and cheering fans, participant spectators, and/or any other entity viewing or participating in the game, on the field of play, in the stands and/or in remote locations to transmit game energy to impact the outcome of the game.

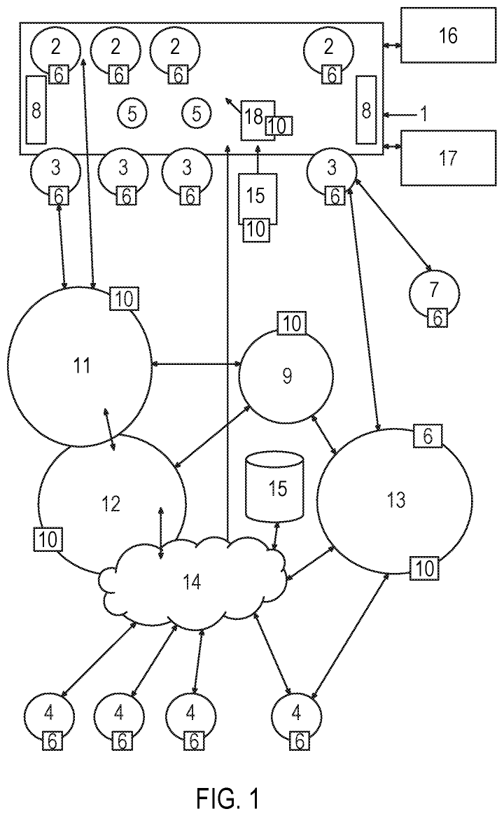

FIG. 1 illustrates a system and associated method for game play management that may be influenced by spectator activity. The system and associated method may be used for promoting physical activity by an individual spectator or group of spectators for a spectatable event. The spectatable event can be one or more games, competitions, and/or events in which outside participation can be and/or is desirable to influence the outcome of the game (hereinafter “game”), in accordance with an exemplary embodiment of the present disclosure. At least one example of the spectatable event includes two teams, each having at least one player playing a game to win. The variable game parameter may further define gameplay for the spectator event by setting e.g., a position of a goal, a change in an obstacle of the game, a movement of an object of the game, a game plan, an offensive game plan, a defensive game plan, a rule of the game, a strike zone, field dimensions, net position, number of players, number of balls or playing objects, number of goals, and/or coaching activity and/or the like. While FIG. 1 illustrates various systems and components, FIG. 1 illustrates one example of a system and associated method of the present disclosure, and additional components can be added and existing systems and components can be removed.

In embodiments, the game can be and/or include one or more real-world games e.g., football, baseball, soccer, hockey, softball, basketball, dodgeball, tennis, pickle ball, track and field, volleyball, golf, board games, casino games, games of chance, game shows (such as TV game shows, e.g., American Idol, Jeopardy, The Price Is Right, etc.), one or more virtual games (e.g., a computer game, online game, or simulation), and/or combinations thereof, further defined to have a variable game parameter. For example, the one or more real-world games and/or virtual games can include sporting event and/or competitions, board game, role playing games, virtual games, fantasy games, virtual reality games and competitions, video games, and the like. In embodiments, the entities associated with the game, as described below, can be real-world persons, virtual avatars (e.g., virtual playable characters), non-playable virtual characters (e.g., players controlled by artificial intelligence), and other types of real-world and/or virtual characters. The entities may or may not operate cooperatively to play the game, whether a real-world game, a virtual game (e.g., a computer game or simulation), and combinations thereof. In embodiments, the game can be and/or include any type of competition, task, challenge, puzzle, etc., in which one or more players compete against one or more players, one or more spectators compete against one or more spectators, one or more teams compete against one or more teams, one or more players compete against artificial intelligence (AI), one or more entities compete against one or more entities, one or more teams compete against AI, one or more players work cooperatively to complete tasks, challenges or puzzle, and/or one or more teams work cooperatively to complete tasks, challenges and/or puzzle, and combination thereof, and further configured to have a variable game parameter. In embodiments, the entities participating in the game can be located in the same location, located in different locations, and combinations thereof. In embodiments, the game can include a single game and/or multiple games, whether real, virtual, and/or combination thereof. In an embodiment, the game can be a combination real-world game and virtual game.

In an embodiment, the game can be a board game, whether real-world, virtual, or combination thereof. In an embodiment, the game can be a role-playing game (RPG), whether real-world, virtual, or combination thereof. In an embodiment, the game can be a massive multiplayer online game (MMO) or a massive multiplayer online role-playing game (MMORPG) whether real-world, virtual, and/or a combination thereof. In an embodiment, the game can be a sports event and/or competition, whether real-world, virtual, or combination thereof. In an embodiment, the game can be a simulation game, whether real-world, virtual, or combination thereof. In an embodiment, the game can be a fantasy game, whether real-world, virtual, or combination thereof. In an embodiment, the game can be an e-sports game, whether real-world, virtual, or combination thereof. In an embodiment, the game can be a fantasy sports game, whether real-world, virtual, or combination thereof. In embodiments, the game can be an immersive simulation-based interactive experience e.g., Madden Football. In an embodiment, the game can be an educational game, whether real-world, virtual, or combination thereof. In an embodiment, the game can be a video game, whether real-world, virtual, or combination thereof. In an embodiment, the game can be a gambling and/or chance game, whether real-world, virtual, or combination thereof. While the above lists various types of games, one skilled in the art will realize that the “game” as described herein can include any type of competition, event, and/or activity. Additionally, while the above list various types of games separately, one skilled in the art will realize that the “game” can include multiple games played together, known or to be developed.

In an embodiment, as illustrated in FIG. 1, methods and systems for providing playing the game includes one and/or more entities that are associated with and/or participate in the game. In embodiments, the one and/or more entities can include one or more of player combatants 11 (offensive player combatants 2, and/or defensive player combatants 3), an individual of the spectatable event e.g., participant spectator(s) 4 or Participant Spectators of Special Interest (PSOSI) 7, fans not affiliated with either or any of the teams that are actively playing, player coaches and/or staff 9, spectator coaches and/or staff 12, cheerleaders 13, judges 16, and/or referees 17. In embodiments, the player combatants 11 can include offensive player combatants 2, and/or defensive player combatants 3.

Participant spectators not affiliated with the home or visiting team and/or teams known as participant spectators of special interest (PSOSI) 7 can apportion their game energy (an example of spectator data, as further discussed below) to one or more teams' defense and/or offense. In embodiments, an entity associated with the game, e.g., player combatants 11 (offensive player combatants 2, and/or defensive player combatants 3), participant spectators 4, player coaches and/or staff 9, spectator coaches and/or staff 12, cheerleaders 13, judges 16, and/or referees 17, can be controlled by, impacted by, manipulated by, or influenced by one another and/or by one or more participant spectators through the application of game energy. In embodiments, an entity associated with the game, e.g., player combatants 11 (offensive player combatants 2, and/or defensive player combatants 3), participant spectators 4, player coaches and/or staff 9, spectator coaches and/or staff 12, cheerleaders 13, judges 16, and/or referees 17 can also generate game energy and apportion their game energy to one or more teams' defense and/or offense. In some embodiments, an entity associated with the game, e.g., player combatants 11 (offensive player combatants 2, and/or defensive player combatants 3), participant spectators 4, player coaches and/or staff 9, spectator coaches and/or staff 12, cheerleaders 13, judges 16, and/or referees 17, can transfer at least a portion of their apportioned game energy to another entity associated with the game.

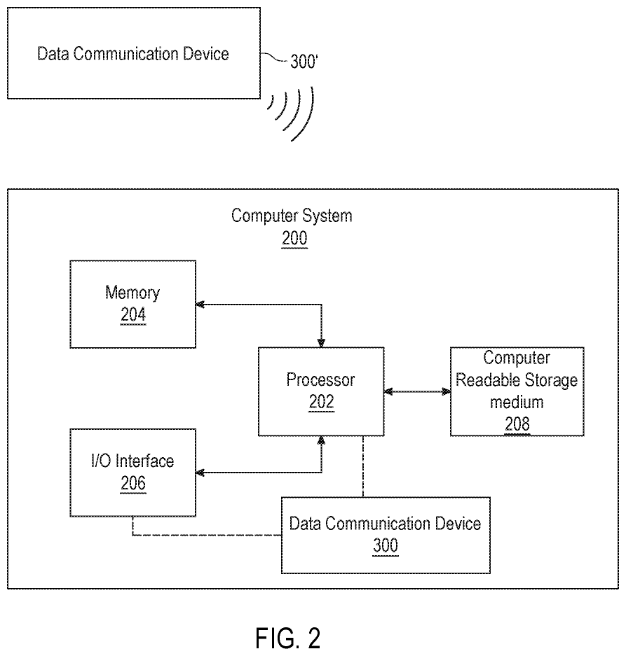

In embodiments, a computing device (e.g., computer system 200 as depicted in FIG. 2) may represent a specific capacity of game energy in the form of one or more integer values. There are a number of ways an entity can generate game energy and participate in the game, for example, by directly inputting the one or more integer values into the computing device, triggering generation of a pre-determined amount of game energy, and/or sending one or more digitized signals (e.g., electro-magnetic signals) via one or more devices (e.g., sensors). The computing device may be configured to convert the one or more digitized signals into the one or more integer value(s) according to a mathematical model or formula. Thus, the computing device may quantify the game energy being generated by participant spectator(s) 4, 7 and then, use that quantified game energy to modify a game parameter, thereby effecting the game play in some manner.

In embodiments, the game can be played by player combatants 11 on the playing field 1 and participant spectators 4, 7 either in the stands and/or in remote locations. As used herein, player combatant 11 refers to an entity, real-world and/or virtual, that is engaged and/or involved in actions of the game. As used herein, a participant spectator 4, 7 refers to an entity, real-world and/or virtual, that observes the game and is involved in the game to potentially influence, impact, alter and/or affect the outcome of the game. In embodiments, all or a plurality of entities related to the game including the field of play and the ball can be real-world and/or virtual. In embodiments, all or a plurality of entities related to the game, player combatants 11, participant spectators 4, 7, and/or other entities, can have a direct influence, impact, and/or effect influence on the outcome of the game. In embodiments, all or a plurality of aspects of the game can be holographic, virtual and/or real including the venue, e. g., stadium. In embodiments, none, some and/or all aspects of the game can be holographic, virtual, and/or real including the venue, e.g., stadium.

In embodiments, the game includes one or more playing objects 5 that are in play and interacted with by the player combatants 11 and/or others, for example, ball, disc, puck and/or energy capsule, that is real, digital, virtual, holographic, and/or any other object. The playing object 5 can include any real, digital, virtual, holographic, and/or the like and/or other objects that a player combatant 11 can handle, toss, carry, throw, kick, pass, shoot, strike, bat, move, send, transmit, block, defend, intercept, steal, hand-off to another player combatant 11, and/or manipulate according to the objectives and rules of the game. In an embodiment, the game can include one or more, or two or more, playing objects 5 that are interacted with by the player combatants 11. For example, the game can include one playing object 5 for each team, where either team can score with either playing object 5.

In embodiments, the game can include one and/or two or more scoring goals 8. Each of the goals 8 can be defended by any of the teams. In embodiments, the goals 8 can be configured in any dimension and/or size that allows the playing object 5 to be received and register a score.

In embodiments, the game can be played both by live in-person offensive player combatants 2 and live in-person defensive player combatants 3. The game can include any number of player combatants 11 as desired. For example, there can be 5 to 25 player combatants per side, and/or less than or more than 5 to 25 player combatants per side, including 1, 2, 3, 4, 5, 6, 7, 8, 9, 10, 11, 12, 13, 14, 15, 16, 17, 18 19, 20, 21, 22, 23, 24, 25, 26, 27, 28, 29, 30 or more.

In another embodiment, there can be less than 10 player combatants 11 per side. In another embodiment, there can be more than 20 player combatants per side, e.g., 30, 40, 50, 60, 70, 80, 90, 100 or more. In embodiments, the players of the two teams can be in the same location. In embodiments, the players of the two teams can be in different locations. In embodiments, the game can include two or more teams, whether located in the same or different locations. In embodiments, the game can exist anywhere in space. In embodiments the game can exist anywhere in real or imagined time.

In embodiments, one or more of the player combatants 11 (offensive player combatants 2 and defensive player combatants 3) can be virtual and/or simulated. For example, the game can be a video game, and the player combatants 11 (offensive player combatants 2 and defensive player combatants 3) can be simulated and/or virtual. In this example, the player combatants 11 (offensive player combatants 2 and defensive player combatants 3) can be controlled by one or more real-world players and/or artificial intelligence and/or influenced by participant spectators and/or the like.

In embodiments, the game can be played by an unlimited number of participant spectators 4, 7 either live and/or remote. The participant spectators 4, 7 contribute their actions such as cheering, noisemaking, exercise, etc., collectively as game play energy, to the play of the game, playing and supporting the one and/or more or the two and/or more teams of the game and/or disrupting the play of the one and/or more or the two and/or more teams of the game. Game play energy is received and collected by the system as spectator data. In embodiments, the game can be played by an unlimited number of teams both live and remote. In embodiments, the game can be played by only one team, live and/or remote with any number of participant spectators, player combatants and/or other entities associated with the game. In embodiments, the game can be played by one team and or two teams and/or more than two teams, either live and/or remote. In embodiments, the participant spectators can influence any and/or all of the teams live and/or remote.

The participant spectators 4, 7, which are on location, can be at the venue of the game, e.g., stadium, field, arena, etc. and optionally their game energy will or will not fully and/or partially be accorded more weight and/or a higher score, less weight and/or a lower score, and/or the same weight and score compared to remote and/or virtual participant spectators and/or the like and/or their scores and/or the generation of game play energy. In one embodiment their game play energy can be handicapped according to the rules of the game. The participant spectators 4, 7, which are remote, can either be at home or another place, either alone or in a group setting. The game can also be played by an unlimited number of participant spectators 4,7 who may be aligned with either of the teams playing both live and remote who contribute their cheering and/or energy to the play of the game. Additionally, the game can also be played by an unlimited number of participant spectators 4,7 who may not be aligned with any team playing both live and remote who contribute their cheering and/or energy to the play of the game. In an embodiment, participant spectators 4, 7, player combatants 11, and any other entity and/or object associated with the game may be real, human, animal, digital, holographic, virtual and/or the like.

In an embodiment, the participant spectators 4, 7 for the offense or defense can connect physically, digitally, electronically, virtually, audibly, and/or the like, whether in person or remote so their actions can be measured, and/or their efforts can be used to contribute their game play energy to one or more teams and/or player combatants 11, for example, their home team, their favorite team, their favorite player, and/or any entity associated with the game according to the rules and objectives of the game. In an embodiment, the participant spectators 4, 7, whether in person or remote, for the offense and/or defense, can generate actions e.g., noise manually and/or with a noise-making instrument. For example, the participant spectators 4, 7 can cheer, clap, chant, stomp, yell, sing, etc, and/or beat a drum, ring a cowbell, blow a horn, play a musical instrument, etc., and the decibels and/or the like can be converted to game play energy according to the rules of the game. In one embodiment, the decibels generated by singing would be converted to game energy based upon factors, such as age, previous performance, wagering, the purchase of game energy enhancements, the guy accuracy of words; the accuracy of tonality; the simultaneous generation of aerobic exercise; the simultaneous generation of anaerobic exercise; a combination of above; and or the like.

The participant spectators' 4, 7 actions directly relate to and/or generate game play energy that directly impacts the game. Additionally, noise generated by the remote participant spectators 4, 7 and/or any entity associated with the game can be transmitted as noise into the stadium. Additionally, game play energy generated by participant spectators 4, 7 and/or any entity associated with the game can be transmitted as noise into the stadium. The participant spectators' efforts, and/or the efforts of any entities associated with the game, can be used to contribute to their home team, their favorite team, their favorite players, and/or the like, according to the rules of the game.

In an embodiment, the participant spectators 4, 7 for the offense and defense can connect digitally, electronically, virtually, audibly, and/or the like, whether in person or remotely located, so that their actions and/or activities can be measured. In an embodiment, the participant spectator actions can also be configured to contribute to the game, conditionally, if it is consistent with their play for the season and consistent with their loyalty to their home team or their favorite team or according to the rules of the game. In an embodiment, the participant spectator's contribution to the game and/or actions can be handicapped according to the rules of the game.

In embodiments, points are awarded in the game when goals are scored and/or according to the rules of the game. The point allocation for scoring may be different for offensive players and/or teams scoring with the teams' offensive ball and/or balls and/or the like compared with their scoring with the other team's ball and/or balls and/or the like and/or according to the objectives and rules of the game. Points can also be scored for a variety of different playing objectives. In embodiments, the playing object and/or objects 5 (ball, disc, puck, energy capsule, ring, and/or any other object) can be real, digital, virtual, holographic, combinations thereof, and/or the like. In embodiments, the goal and/or goals 8 can be real, digital, virtual, holographic, combinations thereof, and/or the like. For example, the playing object 5, whether real, digital, virtual, holographic, and combinations thereof, scores a goal when it gets inside a goal 8. The goal 8 size and shape can be based on the size of the playing object 5. For example, the goal size and shape 8 could be 1 to 3 meters, inclusive, wide and/or long and/deep and/or less than 1 meter or more than 3 meters wide and/or long and/deep and/or the goal size and shape could be 1 to 3 meters in diameter, inclusive and/or less than 1 meter in diameter or more than 3 meters in diameter. In another example, a goal 8 could be the size of a known goal such as the size of the basketball hoop, a soccer goal, a lacrosse goal, a field hockey goal, a baseball strike zone, an ice hockey goal, football goal posts, and/or the like. In one embodiment the goal can exist anywhere in time and space. In one embodiment the goal can be any size and/or shape and/or position and can vary in size and/or shape and/or position, according to the rules of the game. In one embodiment the goal can be any size and/or shape and/or position and can vary in size and/or shape and/or position, according to the game energy generated and/or according to the rules of the game.

In embodiments, the player combatants 11 can play with and/or score with an actual and/or virtual playing object 5. The player combatants 11 can handle, toss, carry, throw, kick, pass, shoot, strike, bat, move, send, transmit, block, defend, intercept, steal, manipulate, transmit, telepathically-transmit, hand-off and/or likewise affect the playing object. For example, the player combatants 11 can handle, toss, carry, throw, kick, pass, shoot, strike, bat, move, send, transmit, block, defend, intercept, steal, manipulate, transmit, telepathically-transmit, hand-off and/or likewise affect the playing object 5 movement and/or the like from one teammate combatant to another teammate combatant 11 and/or from one teammate combatant to another part of the playing field as part of a pre-designed play; as part of the game; as part of an effort to avoid obstacles, to take advantage of obstacles, to avoid members of the other team and/or teams; in an effort to score or prevent a goal or prevent scoring, and/or according to any type of objective and/or rule of the game. The defensive team's combatants 3 can try to steal the one or more playing objects 5 from the offensive team and score those playing objects 5 into the opponent's goal 8. The offensive team's combatants 2 can try to score with their own ball into the opponent's goal. In another example, there are two or more playing objects 5 in play. In this example, each team can have one or more playing objects 5 that they are trying to score into the opponent's goal and/or trying to score in some other manner. Each team and/or any entity involved with the game also tries to steal the opponent's one or more playing objects 5 and score with those playing objects 5, whether real or virtual.

In an embodiment, the goals 8 can be movable across a time and space continuum by the participant spectators 4, 7 and/or by any entity associated with the game. For example, the goals can be movable by the participant spectators 4, 7 and defended by the actions of the opposing team's participant spectators 4,7. The goals 8 can be movable by the participant spectators 4, 7 and defended by the actions of the opposing team's participant spectators 4, 7, through the game play energy generated by participant spectators 4, 7, and/or any other entity associated with the game.

In embodiments, the participant spectators 4,7 and others involved in the game can generate actions through one or more devices, systems, and processes, as discussed below, which are then processed to generate game play energy. As described herein, “game play energy” (or simply “game energy”_refers to a quantifiable measure of the actions and/or activities of a participant spectator 4, 7 (or other entities), which can be utilized by any of the entities associated with the game to affect an aspect or variable of the game. The game play energy or game energy is a form of spectator data. In an embodiment, the participant spectators 4, 7 and/or others involved in the game can generate actions by operating exercise equipment that is located in the stands, on the field, in remote locations, and/or other locations. For example, participant spectator action devices 6 (or simply “devices”) can be configured to track and/or generate actions i.e., game play energy, that include, but are not limited to: riding or pedaling a stationary, spin, or road bicycle; riding or pedaling another type of cycle; walking; walking a dog and the like; hula-hooping; marching; running on a treadmill, track, or the road and/or elsewhere; using a standing or sitting elliptical exercise running machine; batting or swinging a baseball or cricket bat; swimming; rowing; stair climbing; trampolining; bouncing; juggling; water aerobics; aerobic exercise; anaerobic exercise; standing; hopping; dancing; applying pressure, sustaining muscle tension; stretching; performing yoga; doing isometric exercise; doing isotonic exercise; track and field sports including hammer throw, shot put, discus, pole vault, etc.; performing gymnastics; spinning; torquing; turning; jumping rope; hitting a speed bag; hitting a heavy bag; jumping; bouncing a ball; shooting a ball; waving, twisting, rocking on a rocking chair, whipping or twirling a rally towel; upper extremity, lower extremity, upper and lower extremities, and/or rowing ergometers, and/or other types of devices and process that allow user to expend energy. In certain embodiments, the operators and/or owners of the game can provide exercise equipment designed to be used with the game. In embodiments, the operators and/or owners of the game can certify and/or approve of certain exercise equipment.

The devices 6 include computing devices that may be (e.g., communicatively) coupled to the exercise equipment and/or operate as the exercise equipment. In embodiments, some devices 6 may be embedded within or attached to an exterior of the exercise equipment while other devices 6 may be mobile devices used by the participant spectators 4,7 to perform actions for generating game energy. In embodiments, the devices 6 may communicate information indicative of the generated game energy to a second (e.g., stationary) device for storage such as a gaming console (e.g., a computer, a smart TV, a NINTENDO® Switch, the WII®, SONY® PLAYSTATION®, Microsoft® with the Xbox® Series X and/or the like).

As another example, the participant spectators 4,7 operate a smart phone, smart device, smart watch, Wearable System, a wearable and/or the like (with or without additional weight such as from the cover of the smart phone, weighted glove and/or other attachments) as a piece of exercise equipment (e.g., a weight) in an exercise activity (e.g., light weight, high repetition exercise; calisthenics; and/or the like) to create game energy. In some embodiments, the smart phone, smart device, smart watch, Wearable System, wearable and/or the like can be operated similar to a joystick and/or a controller and/or a sensor and/or a joy-con and/or in a similar way to a Wii and/or as a piece of exercise equipment. The smart phone, smart device, smart watch, Wearable System, wearable and/or the like may be equipped with hardware and/or programmed by software for detecting/tracking the exercise activity and capturing any spectator energy created during participant spectator actions. The hardware/software may enable the smart phone to capture information associated with device directionality, speed of the spectator participant and/or speed of the smart phone itself, movement of the smart phone itself, accuracy of the movement of the smart phone itself, location/acceleration/velocity of the smart phone, exercise history, resting calories burned, active calories burned, heart rate, heart rate variability, walking speed, running power, running speed, running stride length, vertical oscillation, flights climbed, stand time, sleep time, exercise time, walking step length, double support time, position, elevation, excursion of the arms, legs, core, head, neck and/or the like, air speed, stair speed down, stair speed up, cardio fitness, ECG, running speed, respiratory rate, noise level, decibel level, facial expressions such as smiling and frowning, VO2 max, blood delusional gain oxygen, DualSense control types of input including gyroscope, shock sensors, haptic feedback, walking steadiness, fitness tracker, weighted controllers, weighted joy-cons and/or the like. In some embodiments, the participant spectator can be equipped with more than one smart phone. In some embodiments, the participant spectator can be equipped with one and/or more than one smart device that can be attached to the participant spectator with a glove, a strap, and/or in a way that measures one or more of these participant spectator activities. In some embodiments, the participant spectator can be equipped with one and/or more smart phones, smart watches, smart devices, wearables, Wearable System and/or the like that measure participant spectators metabolic, cardiovascular activity, game energy generating activity, and/or the like.

In some embodiments, the smart phone, smart device, smart watch, Wearable System, wearable and/or the like can be the exercise equipment and/or the playing object and/or be located inside the playing object and/or be part of the playing object and/or be used by participant spectators, coaching staff, cheerleaders, judges, referees, and/or any other entity associated with the game. In some embodiments the smart phone, smart device, smart watch, Wearable System, wearable and/or the like game energy generating device and/or activity and/or other exercise equipment can directly affect the game energy level of one or more participant spectators, player combatants, coaching staff, cheerleaders, judges, referees, and/or any other entity associated with the game. In some embodiments the movement, actions and/or the like of the participant spectators, coaching staff, cheerleaders, judges, referees, and/or any other entity associated with the game can be tracked and/or monitored by the smart phone, smart device, smart watch, Wearable System, wearable and/or the like and/or the game energy generating equipment for contributing to the participant spectator's game energy.

In some embodiments, the smart phone, smart device, smart watch, Wearable System, wearable and/or the like can be configured e.g., via a software application download or otherwise configured to track the smart phone in six degrees of freedom. In embodiments, the smart phone, smart device, smart watch, Wearable System, wearable and/or the like can be coupled with an attachment to track the smart phone in six degrees of freedom relative to a reference frame e.g., similar to a three-dimensional positioning device that can track the smart phone in space. The smart phone, smart device, smart watch, Wearable System, wearable and/or the like and/or attachments to these devices and/or the like can contain linear accelerometers that measure acceleration along three pairwise orthogonal axes via three linear accelerometers. The accelerometers can directly be used to measure pitch and roll angles, by measuring the direction of gravity, and also form the basis for gesture recognition. In other embodiments, the smart device or attachment can include a gyroscope, an infrared camera, Bluetooth, and/or a sensor bar e.g., four LED clusters. In embodiments, the smart device or attachment can be configured to include motion sensing capabilities similar to a Wii Remote, which allows the user to interact with and manipulate items on screen via motion sensing, gesture recognition, and pointing which is used for a console, using accelerometer and optical sensor technology. The smart device can be another data communication device 300′ for transmitting spectator data to the system e.g., system 200.

Even when the smart device is not used as the weight, the example hardware/software may capture the energy generated by a participant spectator when performing a specific exercise activity. It should be appreciated that other applications are available for the example hardware/software.

In embodiments, physiological data of the entities associated with the game can be measured, such as heart rate including heart rate variability and resting heart rate, blood pressure, respiration rate, caloric intake or use, pulse-oxygen level, temperature, brain wave activity, recovery, muscle tension, oxygen consumption, sweat gland activity, and/or any other type of physiological data and/or biological data. For example, in embodiments, heart rate variability, and resting and/or active heart rate and/or any physiological data can be measured and converted to game energy.

Exemplary devices 6 can include pulse oximeter, a blood pressure meter, an electroencephalogram (EEG), an electrocardiogram (EKG), an electrodermograph, heart rate monitor, a breathing monitor, and the like. In embodiments, fitness can be tracked with electronic fitness training devices such as Fitbit™, WHOOP® Strap™, Spade and Co. Smartwatch™ 2, APPLE® Watch™ Series 7, GARMIN® Vivosmart4™, AMAZON® Halo View™, ERGWALL® Fitness Tracker™; smart phones such as APPLE® iPhone® 14, iPhone® 14 Plus, iPhone® 14 Pro, iPhone® 14Pro Max,; Google Pixel, Samsung Galaxy Note, Samsung, android phones, iOS phones, and/or the like, for tracking participant spectator game energy, and/or other aspects of the game. That is, exemplary devices 6 for tracking participant spectator and other entities associated with the game actions and game energy, and/or other aspects of the game can be any device that measures actions, fitness, and/or activities of a user, for example, similar to and/or different from when a user is performing an exercise activity (e.g., calisthenics). That is, exemplary devices 6 for tracking participant creation of noise, music and or singing in the furtherance of creating game energy, and/or to affect other aspects of the game can be any device that measures the generation of noise, music, singing and or sound of a user. For example, the generation of noise, music, singing and or sound of a user can be similar to and/or different from the generation of noise, music, singing and or sound of a user that is coordinated with an exercise activity (e.g., aerobic activity) and or the generation of noise, music, singing and or sound of a user that is in consonance and or dissonance with other entities associated with the game. Some exemplary devices 6 may be configured to operate as an input/output device (e.g., as a controller and/or as a joystick) for enabling entity participation in the exercise activity.

In other examples, actions applicable to generate game energy can include various aspects of the game, including, but not limited to the speed and/or elapsed time of getting the ball from the stands onto the field of play, and other actions associated with a particular game e.g., defensive actions, offensive actions, set up actions, buying actions, selling actions, movement actions etc. In other examples, exemplary actions applicable to generate game play energy can include but are not limited to cross country skiing; downhill skiing; telemark skiing and/or other skiing or snowshoeing; stair climbing; rock climbing; gambling; spending money; tokens; cryptographic tokens; and/or any type of currency; kicking a ball; swinging a racquet; swinging a golf club; doing physical exercise activities e.g., push-ups, sit-ups, pull-ups, chin-ups, jumping jacks, squats, and/or other similar exercises; weightlifting; creating alpha, beta, theta or delta brain-waves, and/or exercise or movement of any other kind in any place including movement while seated and/or in a specially designed stadium chair and/or high-intensity interval training (HIIT); and/or exercise classes like vigorous step aerobics and/or kickboxing; and/or muscle-strengthening activities including lifting weights, working with resistance bands, calisthenics, doing calisthenics that use body weight for resistance (such as push-ups, pull-ups, and/or planks); carrying heavy loads; and/or heavy gardening; and the like.

In embodiments, actions applicable to generate game play energy can include exercises performed on devices that provide and/or measure aerobic exercise, anaerobic, and/or muscular strengthening exercises. Applicable exercises can be performed on devices that provide and/or measure exercise, muscle strengthening, and/or bone strengthening. In embodiments, exercise can be performed on proprietary equipment that can be specifically designed for the game to provide one or more types of exercise. For example, stadium chairs and/or other seats themselves, or the floor under the chairs may be configured to include sensors to sense the actions of a participant spectator e.g., their movement such as the torque of the participant spectators who are sitting in stadium chairs, standing, jumping, and/or the like. In embodiments, the operators and/or owners of the game can provide applicable exercise equipment for generating and/or measuring and processing game energy. In embodiments, the operators and/or owners of the game can certify and/or approve certain exercise equipment. In embodiments, heart rate variability trainers and/or resting heart rate trainers and/or the like can be used.

Participant spectators' 4, 7 actions and the actions of others involved in the game may be measured based on the strength, speed, distance traveled, accuracy, and/or other metrics related to the rules and/or objectives of the game, both defensively and/or offensively and can be converted to game play energy. Participant spectators' 4, 7 actions and the actions of others involved in the game may be measured based on physiological parameters such as heart rate, blood pressure, respiration rate, caloric intake or use, pulse-oxygen level, temperature, brain wave activity, recovery, muscle tension, oxygen consumption, sweat gland activity, and/or any other type of physiological data and/or biological data. Speed, velocity and/or acceleration can be measured e.g., via sensors, in revolutions per minute (RPM), meters per hour, kilometers per hour, feet per second, miles per hour, feet per second per second, and/or any other measurement of displacement over time. Strength may be measured by resistance on the exercise device and/or via other measures and/or on any device. Acceleration may be measured by resistance on the exercise device and/or via other measures and/or on any device. Distance may be measured in miles, kilometers, and/or any other measurement of displacement. Accuracy and the like can be measured according to goals scored and/or assisted, saves made and/or assisted plays demonstrating skill, strength, finesse, expertise, planning and/or the like, and/or other metrics related to the rules and/or objectives of the game, both defensively and/or offensively. The participant spectators 4, 7 energy and/or the energy generated by others can be applied to the outcome of a play and/or game and/or any aspect of the game. In embodiments, the measurement of the participant spectators' 4, 7 actions or activities and/or the energy generated by others can be converted into a value of game play energy i.e., a form of spectator data.

In embodiments, game energy can also be generated based on the quality and/or quantity of noise level inputs and/or outputs e.g., decibels of noise generated and/or some other way to measure the noise generated as a result of singing, chanting, cheering, praying; clapping, banging, booing, hissing, whistling, screaming, and/or some other game-related generated noise, and/or activity and/or as a result of the decibels of noise generated and/or some other way to measure the noise generated using noise makers or other machines, instruments, and/or apparatus to generate noise and/or music.

In embodiments, with respect to the combatant players 11, game energy generation by any entity associated with the game can be utilized for, but not limited to, making plays, blocking goals, cheering on the participant fans, scoring goals, executing plans, executing plays, executing strategies and/or for blocking, preventing goals, preventing being scored upon and/or executing plans, executing plays, executing strategies that prevent being scored on, facilitating stealing the opponent's real and/or virtual playing object 5, and/or other metrics associated with the rules and objectives of the game.

In embodiments, the participant spectators and/or other entities associated with the game 4, 7 can also generate game play energy through virtual game and/or real game and/or any type of competition between participant spectators and/or others. In embodiments, the participant spectators 4, 7 can also generate game play energy through exercise actions and/or the creation of noise in response to a game plan. In embodiments, the participant spectators 4, 7 can also generate game play energy through exercise and/or the creation of noise in response to direction from the cheerleaders 13. In embodiments, the participant spectators 4, 7 can also generate game play energy through exercise and/or the creation of noise in response to signals from the coaches 9 and/or player combatants 11. In embodiments, the participant spectators 4, 7 can also generate game play energy through exercise and/or the creation of noise in response to a selected play. In embodiments, the participant spectators 4, 7 can also generate game play energy through exercise and/or the creation of noise in response to the game. In embodiments, any entity associated with the game can generate game play energy that impacts or influences a variable game parameter e.g., supports, hinders, is involved with, and/or enhances game play, upon reaching predetermined threshold value of the variable game parameter. A threshold value of variable game parameters can be e.g., a predetermined game energy level, a predetermined noise level, a predetermined number of users exercising, or a predetermined level of a quantifiable measure of the actions and/or activities of the participant spectator, and the like.

According to another embodiment, the participant spectators 4, 7 can also generate game energy through exercise and/or the creation of noise in response to the release of playing object or objects 5 into the area of the player combatants 11, into the area of the participant spectators, into the maze, into a portion of the field from which the playing object can gain entry to the playing field, and/or into another area of competition. In embodiments, the participant spectators 4, 7 may also generate game play energy through exercise and/or the creation of noise in response to the release of playing object or objects 5 from the maze from which it can be released onto the field of play and/or into the area of the participant spectators 4, 7. In embodiments, the participant spectators 4, 7 may also generate game play energy through exercise and/or the creation of noise in accordance with the rules of the game. In embodiments, the participant spectators 4, 7 and/or any entity associated with the game may also generate game play energy through exercise and/or the creation of noise in accordance with the rules of the game.

In embodiments, the participant spectators may generate game play energy through exercise and/or the creation of noise and the like. In embodiments, the quantity of the game play energy output from each participant spectator 4, 7 can be related to the actions from the participant spectator exercising on regulated and/or proprietary and/or any exercise equipment located in a stadium and/or venue or remotely. The game play energy can be generated to manipulate and/or impact a variable game parameter of the game. This promotes the amount of exercise and/or the amount of game energy generation of the participant spectators 4,7 while they watch, and/or while the participant spectators 4, 7 physically and/or emotionally participate in the game and/or this promotes the likelihood of the participant spectators 4,7 exercising and/or generating game energy while they watch, and/or while the participant spectators 4, 7 physically and/or emotionally participate in the game. The amount of game play energy generated can be related to and/or based on the amount of energy expended by the participant spectator 4, 7 in connection with a particular action e.g., actions on a particular equipment. Each individual participant spectator 4,7 can contribute to the effort of their team. Each individual participant spectator 4, 7 can contribute to the outcome of the game. Any entity associated with the game can contribute to the outcome of the game by creating and/or distributing game energy according to the rules of the game.

The system for game play has its processor further configured to output to the participant spectator via a display or other communication device, a winner of the spectatable event and spectator data analytics. The data analytics can include, e.g., the type of spectatable event, the number and type of participant spectators participating in the spectatable event, the number and type of entities participating in the spectatable event, the various variable game parameters of the spectatable event, the spectator data, the predetermined threshold values of the variable game parameters, etc. and/or combinations thereof.

In embodiments, the electronic and computer systems, e.g., the participant spectator action devices 6, can be coupled to a communication network 14 and a computer system that includes a server 15, and a processor. The server 15 can be configured to receive the measurements of the actions and/or activities of the entities associated with the game, participant spectators 4, Participant Spectators of Special Interest (PSOSI) 7, player coaches and/or staff 9, spectator coaches and/or staff 12, cheerleaders 13, judges 16, referees 17, and/or other entities associated with the game) and generate values for the game play energy corresponding to the measured activity i.e., game energy data. The server 15 can store and utilize one or more algorithms, conversion tables, and like that map and convert the measured actions and/or activities (game energy data) to a value of game play energy. The server 15 can generate and maintain records that store the values of game play energy with links that reference the corresponding entities and/or teams. The server 15 can also maintain records and rules that define how game play energy can be used to affect the game. During the game, the server 15 can provide indications of the generated game play energy to any of the entities that may utilize the game play energy to affect the game, e.g., coaches 9. If an entity selects to use an amount of the game play energy to affect the game, the server 15 can send a command, via the communications network 14, that causes an action to be performed and can update the record of the game play energy used. For example, if a coach 9 selects to move a goal 8, the server 15 can send a command to move the goal 8 and reduce the game play energy by a predetermined amount in the records.

The electronic and computer systems, the communication network 14, and the server 15 can communicate via one or more communication links. The communication links can be direct or indirect. A direct link can include a link between two devices where information is communicated from one device to the other without passing through an intermediary. For example, the direct link can include a Bluetooth™ connection, a Zigbee™ connection, a Wifi Direct™ connection, a near-field communications (NFC) connection, an infrared connection, a wired universal serial bus (USB) connection, an ethernet cable connection, a fiber-optic connection, a firewire connection, a microwire connection, and so forth. In another example, the direct link can include a cable on a bus network. “Direct,” when used regarding the communication links, can refer to any of the aforementioned direct communication links.

An indirect link can include a link between two or more devices where data can pass through an intermediary, such as a router, before being received by an intended recipient of the data. For example, the indirect link can include a wireless fidelity (WiFi) connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (GSM) standard, a code division multiple access (CDMA) standard such as the universal mobile telecommunications standard, an orthogonal frequency division multiple access (OFDMA) standard such as the long-term evolution (LTE) standard, and so forth. “Indirect,” when used regarding the communication links, can refer to any of the aforementioned indirect communication links.

In embodiments, electronic and computer systems can include data communication capabilities via data communication devices 300. Such capabilities can be rendered by various electronics/devices for transmitting and/or receiving electronic and/or electromagnetic signals. For example, electronic and computer systems can include one or more data communication devices. The data communication device can include, for example, a networking chip, one or more antennas, and/or one or more communication ports. The data communication device can generate radio frequency (RF) signals and transmit the RF signals via one or more of the antennas. The data communication device can receive and/or translate the RF signals. The data communication device can receive the RF signals. The RF signals can be broadcast and/or received by the antennas. The data communication device can generate electronic signals and transmit the RF signals via one or more of the communication ports. The data communication device can receive the RF signals from one or more of the communication ports. The electronic signals can be transmitted to and/or from a communication hard line by the communication ports. The data communication device can generate optical signals and transmit the optical signals to one or more of the communication ports. The data communication device can receive the optical signals and/or can generate one or more digital signals based on the optical signals. The optical signals can be transmitted to and/or received from a communication hardline by the communication port, and/or the optical signals can be transmitted and/or received across open space by the networking device. The data communication device can also receive and/or output audio signals. The data communication device can generate one or more digital signals based on the audio signals that can be output and/or transmitted to other devices.

The data communication device 300 can include hardware and/or software for generating and communicating signals over a direct and/or indirect network communication link. For example, the communication component can include a USB port and a USB wire, and/or an RF antenna with Bluetooth™ programming installed on a processor, such as the processing component, coupled to the antenna. In another example, the communication component can include an RF antenna and programming installed on a processor, such as the processing device, for communicating over a Wifi and/or cellular network. As used herein, “communication device” “communication component,” and/or “communication unit” can be used generically herein to refer to any or all of the aforementioned elements and/or features of the communication component.

The server 15 can include a physical server and/or a virtual server. For example, the server device can include one or more bare-metal servers. The bare-metal servers can be single-tenant servers or multiple tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers. The term server can refer to functionality of a device and/or an application operating on a device. For example, an application server can be programming instantiated in an operating system installed on a memory device and run by a processing device. The application server can include instructions for receiving, retrieving, storing, outputting, and/or processing data. A processing server can be programming instantiated in an operating system that receives data, applies rules to data, makes inferences about the data, and so forth. Servers referred to separately herein, such as an application server, a processing server, a collaboration server, a scheduling server, and so forth can be instantiated in the same operating system and/or on the same server device. Separate servers can be instantiated in the same application or in different applications.

In embodiments, the electronic and computer system can be physical computer devices, an example of which is described below in FIG. 2, or a general-purpose computer configured to or having software or computer executable instructions to receive actions input from participant spectators and/or other entities associated with the game, determine game play energy based on the actions, and manipulate the game based on the determined game play energy. In embodiments, the computer devices can include cloud computing services. As used herein, a “cloud” or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

Various aspects of the systems described herein can be referred to as “data.” Data can be used to refer generically to modes of storing and/or conveying information. Accordingly, data can refer to textual entries in a table of a database. Data can refer to alphanumeric characters stored in a database. Data can refer to machine-readable code. Data can refer to images. Data can refer to audio. Data can refer to, more broadly, a sequence of one or more symbols. The symbols can be binary. Data can refer to a machine state that is computer-readable. Data can refer to human-readable text.

In embodiments, one or more devices measure physiological measurements of the entities associated with the game and communicate the measurements to the server 15. The server 15 can convert the measurements to game energy. Devices 6 will transmit information into the hardware system, operating system or components thereof and communicate to the server 15.

Further, in embodiments, the participant spectators 4, 7 either in-stadium or remotely can be organized into pre-determined groups of participant spectators 4, 7 of a set number and/or indeterminant number per group and/or location. For example, the participant spectators' 4, 7 actions are combined to contribute to the efforts of their team. The contribution of each individual participant spectator 4,7 and/or each group of participant spectators 4, 7 can be recorded and/or acknowledged. For example, the participant spectators 4, 7 with the actions resulting in the most game play energy production and/or with the most effective use of their game play energy production may, with their permission, be identified as such. For example, the participant spectators 4, 7 with the actions resulting in none and/or any amount of play energy production and/or none and/or perfect and/or ideal and/or exemplary use of their game play energy production may, with their permission, be identified as such. In another example, the participant spectators 4,7 and/or other entities associated with the game with the most effective use of their game play energy production to help their offensive team may, with their permission, be identified as such. In another example, the participant spectators 4,7 and/or other entities associated with the game with the most effective use of their game play energy production to help their defensive team, may, with their permission, be identified as such. In another example, the participant spectators 4,7 and/or other entities associated with the game with the most effective use of their game play energy production to help their offensive team and/or defensive team may, with their permission, be identified as such. In another example, the participant spectators 4,7 and/or each group of participant spectators 4, 7 and/or other entities associated with the game with the most effective use of their game play energy production to help their offensive team and/or defensive team and/or according to the rules of the game may, with their permission, be identified as such, and/or earn prizes, equipment, tickets, tokens, money, honors, contracts and/or the like. Participant spectators being part of a group of any size can promote a feeling of social connection, community and belonging. Participant spectators, i.e., spectators who are participating, by identifying and supporting a team through the participant spectator's generation of game energy will be promoting their own feeling of social connection, community and belonging. In an embodiment, any of the participant spectators, with their permission, may be identified.

In embodiments, at the stadium, arena, and/or venue, box seats or other types of seating can have devices for monitoring actions used to generate game play energy (e.g., exercise equipment and/or noise-making equipment and/or musical instruments and/or the like) that more than one person may operate separately and/or together and/or one or more pieces of exercise equipment and/or noise making equipment may be shared by those seated in the box. Remote participant spectators 4, 7 can also share a piece of energy-generating and/or noise-making equipment. Sharing equipment can promote social connection, community and a sense of belonging.

In embodiments, fouls can result in penalty time and/or reduction of game energy and/or temporary reduction of the ability to generate game energy. For example, penalty time can be resolved by placing the playing object 5 back into the crowd to be re-launched into the field of play either by passing the real or virtual playing object and/or objects 5 through the stands and/or through a maze. In embodiments, referees 17 may penalize players and teams and stop and/or start play according to the rules of the games. For example, referees 17 can penalize player combatants 11; participant spectators 4,7; coaches 9, 12; and/or cheerleaders 13 in a variety of ways including depleting them of energy. In another example, referees 17 can penalize player combatants 11; participant spectators 4,7; coaches 9, 12; and/or cheerleaders 13 and/or any entity associated with the game in a variety of ways including taking the playing object and/or objects 5 out of play and/or making it harder for any entity associated with the game to generate energy and/or fully or partially deplete any entity associated with the game of its game energy. In another example, referees 17 can penalize player combatants 11; participant spectators 4, 7; coaches 9, 12; and/or cheerleaders 13 in a variety of ways including taking the playing object(s) 5 out of play and putting the playing object temporarily out of play or into the stands and/or for the game to be temporarily halted and/or according to the rules of the game.

In embodiments, the judges 16 can reward teams and players for outstanding performances. For example, judges 16 can reward player combatants 11; participant spectators 4, 7; coaches 9, 12; and/or cheerleaders 13 in a variety of ways including giving them a bonus of energy and/or a monetary bonus based on game play and/or game play energy. In another example, judges 16 can reward player combatants 11; participant spectators 4, 7; coaches 9, 12; and/or cheerleaders 13 in a variety of ways including giving them a bonus of game play energy for the execution of a set play and/or give them a bonus and/or deduction for any play considered worthy of a bonus and/or a deduction. In another example, judges 16 may reward player combatants 11, participant spectators 4,7, coaches 9, 12, and/or cheerleaders 13 in a variety of ways including giving them a bonus of energy and/or money based on game play energy for the execution of an innovative play or part thereof. In another example, judges 16 can reward player combatants 11 in a variety of ways for a variety of game playing including e.g., giving them a bonus of game play energy for scoring from an elevation above the playing field 1 either as result of jumping, climbing and/or leaping individually or as a result of jumping, climbing and/or leaping with the help of others and/or the like. In another example, judges 16 can reward cheerleaders 13 in a variety of ways including giving them a bonus of energy and/or money, for instance, based on game play, game play energy, for generating energy and/or generating noise.

In another example, judges 16 can reward player combatants 11 in a variety of ways including giving them a bonus of energy and/or money based on game play energy generated and/or for scoring from an elevation above the playing field with the help of others and/or according to the rules of the game. In another example, judges 16 can reward player combatants 11 in a variety of ways including giving them a bonus of energy and/or money based on game play energy for scoring from an elevation above the playing field by climbing on others before scoring. In another example, judges 16 can reward player combatants 11 or participant spectators 4,7 in a variety of ways including giving them a bonus of energy and/or money based on game play energy for the execution of a set play. In another example, judges 16 can reward player combatants 11, participant spectators 4,7, and/or any entity associated with the game in a variety of ways including giving them a bonus of energy and/or money based on game play energy for the execution of a set play even though they have given advanced notice to the other team and/or to any other entity associated with the game that they are going to execute that play. In another example, judges 16 can reward player combatants 11, participant spectators 4,7, and/or any entity associated with the game in a variety of ways including giving them a bonus of energy and/or game play energy and/or money based for an outstanding play, effort, teamwork, sportsmanship, enthusiasm and/or the like, and/or penalize them for the lack of these skills, traits, and/or the like and/or according to the rules of the game.

In embodiments, the entities associated with the game, e.g., player combatants 11 (offensive player combatants 2, and/or defensive player combatants 3), participant spectators 4, player coaches and/or staff 9, spectator coaches and/or staff 12, cheerleaders 13, judges 16, and/or referees 17, and/or other entities associated with the game can communicate with each other to play, plan, direct, and/or participate in the game. In embodiments, participant spectators 4, 7 can be encouraged by cheerleaders 13, players, coaches and/or any other entity associated with the game as to when to produce and/or when, where, why and/or how to release game play energy.

In embodiments, game play energy generated by participant spectators 4, 7 can be organized by the spectator coaching staff 12 and/or any other entity associated with the game that coordinates with the player coaching staff 9 and/or any other entity associated with the game to impact or influence a variable game parameter e.g., actually move the goal or goals 8, change the natural and artificial obstacles and/or the conditions that can affect play on the field, direct, hinder, and/or enhance the movement of player combatants 11, direct, hinder, and/or enhance the movement of the ball, disc, puck, energy capsule 5 and/or the like, execute a game plan to score offensive goals and defend against goals being scored by the opponent, and/or other activity associated with the game. The in-person and remote spectator participants 4, 7 can have visual, verbal, digital, and/or otherwise signaled access to and/or from their spectator coaching staff 12 for direction as to how to manipulate the field 1, so as to improve defensive and/or offensive positioning. The player combatants 11 can have their own coaching staff 9 that coordinates with the participant spectators' 4, 7 coaching staff 12. The input of game play energy from the spectator participants 4, 7 both in-house/live and remote can be managed by the spectator coaches 12 if and only if the spectator participants 4, 7 allow their input to be managed. In embodiments the spectator participants 4, 7 can attempt to manipulate the playing field on their own using generated game play energy. Spectator participants 4, 7 can determine a percentage of their game play energy they may control on their own, that they may give to the coaches, and/or that they may proxy to another participant spectator, to a participant spectator group, and/or to any other selection, and/or any other entity associated with the game. In certain embodiments the spectator participants 4,7 can store their energy and/or release their energy as they see fit.

In embodiments, the amount of game play energy required for each specific manipulation of the activity on the field can be known or pre-determined. In embodiments, the amount of game play energy required for each specific manipulation of the activity on the field may not be known and may be determined through a mathematical model or formula. In embodiments, all or part of the amount of game play energy generated by a team is known by that team and/or the other team and/or by player coaches, spectator coaches, cheerleaders, players, spectators and/or any entity associated with the game. In embodiments, all or part of the amount of energy generated by a team may not be known by that team, and/or the other team and/or by player coaches, spectator coaches, cheerleaders, players, spectators and/or any entity associated with the game.

According to an embodiment, game play energy is based on actions generated using energy generation/controlling/distribution devices 6 and/or by creating noise, music, and/or song and/or the measurement of any physiological and/or metaphysical activity. The game energy and/or actions can then be stored, recorded, transferred, transmitted, and/or received via collection/distribution/receiving devices 10 to generate game play energy which can be used to manipulate, affect, influence, alter and/or impact the game. Participant spectator actions can also generate game play energy by online game play as a result of winning and/or playing online computer games and/or parts thereof. For example, the game energy and/or actions of participant spectators can be collected by spectator coaches 12, player coaches 9, cheerleaders, player combatants 11, and any other entity associated with the game. In another example, the game energy and/or transmitted actions can be collected and/or measured by any or all of the following: players, player coaches, spectator coaches, cheerleaders, and participant spectators. In embodiments, the game energy and/or actions can be transmitted to the coaches and coaching staff and/or directly to the players or cheerleaders. The game energy and/or transmitted actions can be collected by player coaches, spectator coaches, cheerleaders, players, participant spectators, and/or any entity associated with the game.

In embodiments, the actions from players, coaches, participant spectators, cheerleaders, and/or any entity associated with the game can be used to generate game play energy for one and/or several variable game parameters/activities, including but not limited to, moving a digital, virtual, holographic, real, 2-dimensional or 3-dimensional goal or digital goals from one position on the scoreboard to another position on the scoreboard; moving, opening or closing a real, digital, virtual, holographic, 2-dimensional or-3 dimensional goal; moving the goal line, moving bases, moving the finish line, moving the passing zone, moving the strike zone, moving the hoop, moving the goal post, moving the net, moving player coaches, spectator coaches, cheerleaders, players, spectators, judges, and/or referees and/or any entity associated with the game and/or for scoring in the game and/or in furtherance and/or in opposition to any of the rules of the game. In embodiments, the actions from players, coaches, participant spectators, cheerleaders, and/or any entity associated with the game can be used for cheerleaders to physically move better and/or faster, to be heard better and/or to sing louder and/or be able to perform in a way that promotes the participant spectators to cheer more and/or louder and/or for the player combatants to perform better. In embodiments, the actions from coaches, participant spectators, cheerleaders, and/or any entity associated with the game can be used for moving a real and/or virtual defender on and/or in front of the scoreboard and/or in front of the moving goal; for freezing, moving, prevent any and/or all movement and/or hindering the movement of the ball; for freezing players to the floor, and/or moving and/or hindering the movement of players; for moving the real and/or virtual playing object and/or objects 5; for moving the player combatants; for moving/changing obstacles, for lifting goals out of the floor; for moving goals along the floor; for moving goals in the air; for moving goals in 3-dimensional space; for players to move themselves; for cheerleaders to move; and/or for any, some or all of the player coaches, spectator coaches, cheerleaders, player combatants, and participant spectators and/or any entity associated with the game to help with any and/or all aspects of the game, including, but not limited to, the scoring of goals, the prevention of goal scoring, the execution of outstanding plays and/or the prevention of the execution of outstanding plays, the execution of plays and/or the prevention of the execution of plays, and/or cheering for any and/or all of these activities and the like according to the objectives and rules of the game, and/or for any entity to move in any dimension in time and/or space and/or according to the rules of the game. Throughout the course of the game, the spectator data and/or game energy is used to update the variable game parameters according to the rules of the game and/or likewise.

In embodiments, the actions from player combatants can be used to generate game play energy for several variable game parameters/activities, including but not limited to, moving a digital, virtual, holographic, real, 2-dimensional or 3-dimensional goal or digital goals from one position on the scoreboard to another position on the scoreboard; moving, opening or closing a real, digital, virtual, holographic, 2-dimensional or-3 dimensional goal, moving the goal line, moving bases, moving the finish line, moving the passing zone, moving the strike zone, moving the hoop, moving the goal post, moving the net, and/or moving player coaches, spectator coaches, cheerleaders, players, spectators, judges, and/or referees and/or any entity associated with the game and/or scoring in the game; for cheerleaders to physically move better and/or faster, to be heard better and/or to sing louder and/or be able to perform in a way that promotes the participant spectators to cheer more and/or louder and/or for the player combatants to perform better; for moving a real and/or virtual defender on and/or in front of the scoreboard and/or in front of the moving goal; for freezing, moving and/or hindering the movement of the ball; for freezing players to the floor, prevent any and/or all movement and/or moving and/or hindering the movement of players; for moving the real and/or virtual playing object and/or objects 5; for moving the player combatants; for moving/changing obstacles, for lifting goals out of the floor; for moving goals along the floor; for moving goals in the air; for moving goals in 3-dimensional space; for players to move themselves; for cheerleaders to move; and/or for any, some or all of the player coaches, spectator coaches, cheerleaders, player combatants, and participant spectators to help with any and/or all aspects of the game, including, but not limited to, the scoring of goals, the prevention of goal scoring, the execution of outstanding plays and/or the prevention of the execution of outstanding plays, the execution of plays and/or the prevention of the execution of plays, and/or cheering for any and/or all of these activities according to the objectives and rules of the game, and/or for any entity to move in any dimension in time and/or space and/or to affect any aspect of the game according to the rules of the game. Throughout the course of the game, the spectator data and/or game energy is used to update the variable game parameters according to the rules of the game and/or likewise.

In one embodiment, the playing conditions can be adjusted by turning on fans to simulate wind that can be used to help or hinder the play, and/or to turn on sprinklers that can simulate rain, simulate wet and/or damp conditions, temperature change, and/or adjust the light, darkness, simulate sunlight, simulate glare, affect gravitational forces, affect time, space and/or other dimensions and/or the like to help and/or hinder the play. In embodiments, other in-person spectators 4 can contribute to the offensive or defensive game plan and/or according to the objectives or rules of the game by expertly executing their portion of the game on a computer or similar device, a game, a virtual reality device, an online game, fantasy game, and the like.

In embodiments, the goals, game conditions, and/or the playing field, etc., can be in a constant state of flux or change. In embodiments, game plans, or strategies may or may not need to be in a constant state of flux and the spectator coaches 12 and/or player coaches 9 may or may not need to work with the player combatants 11 and some and/or all of the participant spectators 4, 7 and/or cheerleaders help to score goals for the offense and with some or all of the participant spectators to prevent goals by helping the defense. In embodiments, the game plan strategy can be directed by the coaches 9 of the player combatants 11 and/or the coaches 12 of the participant spectators 4. The combatant players 11 can physically move the playing object and/or objects 5 to score goals, to execute plays, to execute game plans, and/or to execute other activities associated with the game. The participant spectators 4, 7 can use their actions to move the playing object and/or objects 5 to score goals, and/or assist in the scoring of goals to help the offense and/or other activities associated with the game. The movement of the playing object and/or objects 5 can be controlled by game play energy, other aspects of the game, and the like. Further, the movement, aperture, position, and/or other aspects of the goal and/or goals 8 can be controlled by game play energy, other aspects of the game, and/or the like. The defensive participant spectators can use their actions to block the movement of the playing object 5 to prevent the scoring of goals and/or other types of scoring and/or assist to prevent the execution of plays. The offensive participant spectators can use their actions to assist the movement of the playing object and/or objects 5 to help with scoring of goals, the execution of plays, and/or other activities of the game. Further, the movement, aperture, position, and/or other aspects of the goals 8 can be controlled in a way that helps the defense and/or offense. For example, there can be up to six positions in which the goal or virtual goal can be positioned, placed, and/or moved. As discussed above, the goal refers to any digital, virtual, holographic, real, 2-dimensional, or 3-dimensional goal through which, at which, or near which, the playing object 5 passes or travels for the purpose of scoring or being awarded points. In another example, there can be up to nine positions in which the goal or virtual goal can be positioned, placed, and/or moved. In another example, there can be up to sixteen positions in which the goal or virtual goal can be positioned, placed, and/or moved. In another example, there can be up to twenty-five positions in which the goal or virtual goal can be positioned, placed, and/or moved. In another example, there can be up to thirty-six positions in which the goal or virtual goal can be positioned, placed, and/or moved. In another example, there can be up to forty-nine positions in which the goal or virtual goal can be positioned, placed, and/or moved. In another example, there can be up to sixty-four positions in which the goal or virtual goal can be positioned, placed, and/or moved. In another example, there can be up to eighty-one positions in which the goal or virtual goal can be positioned, placed, and/or moved. In another example, there can be up to one hundred positions in which the goal or virtual goal can be positioned, placed, and/or moved. In another example, there can be one hundred or more than one hundred positions in which the goal or virtual goal can be positioned, placed, and/or moved. In another example, there can be an infinite number of positions in time, space, and/or any dimension where the goal or virtual goal and/or strike zone and/or hoop and/or scoring and/or scoring position goal line and/or base or bases can be and/or any other entities associated with the game can be positioned, placed, or moved.

In embodiments, the goal 8 can be a virtual and/or simulated that includes two-dimensional representation displayed on a computer screen, a virtual headset, and/or other types of display devices. In embodiments, the goal 8 can be a virtual and/or simulated that includes three-dimensional representation and/or hologram displayed in the real world and/or on a computer screen, a virtual headset, and/or other types of display devices. In embodiments, the goal 8 can be an actual hole in the backboard, computer screen, and/or other structure through which the playing object 5 can pass. In embodiments, the goal 8 can be an actual hole in the backboard, computer screen, and/or other structure through which the playing object 5 can pass into the area occupied by the participant spectators 4, 7. In embodiments, the goal 8 can be an actual hole in the backboard, computer screen, and/or other structure through which the playing object 5 can pass into the area occupied by the participant spectators 4, 7 and play begins when the ball is located in the area of participant spectators. In embodiments, the size of the goal 8 can be variable and is to be determined by the rules of the game.

In embodiments, the movement of the goaltender may be controlled by the goaltender himself, herself, themself and/or themselves as well as through the game play energy generated by participant spectators 4, 7 and/or other entities associated with the game. In embodiments, the goal 8 and/or virtual goal 8 can be positioned, placed, and/or moved by the game play energy generated by the participant spectators 4, 7 and/or the player combatants, the coaches 9, the cheerleaders 13, and/or other entities associated with the game. In embodiments, the player combatants play the game and the participant spectators influence the play of the player combatants. In embodiments, the player combatants play the game and the participant spectators influence the play of the player combatants through the generation of game energy In embodiments, the goal 8 and/or virtual goal 8 can be opened and/or closed manually by the offensive player combatants. In embodiments, the goal 8 and/or virtual goal 8 can be opened and/or closed manually by the defensive player combatants. In embodiments, the goal and/or virtual goal can be opened and/or closed manually by the offensive and/or defensive player combatants, and/or other entities associated with the game. In embodiments, the goal 8 and/or virtual goal 8 can be opened and/or closed by the energy generated by the participant spectators 4, 7. In embodiments, the goal 8 and/or virtual goal 8 can be opened and/or closed by the noise generated, as described above, by the participant spectators 4, 7. In embodiments, the goal and/or virtual goal can be opened and/or closed by the energy generated by the participant spectators and/or the player combatants. In embodiments, the goal 8 and/or virtual goal 8 can be opened and/or closed by the game play energy generated by the participant spectators 4, 7, the player combatants 11, the coaches 9, the cheerleaders 13, and/or other entities associated with the game.

In embodiments, according to the rules of the game, any aspect associated with the game can be adjusted as a result of the game play energy generated by the participant spectators 4, 7, the player combatants 11, the coaches 9, the cheerleaders 13, and/or other entities associated with the game.

In one embodiment, each team may have more than one goal 8. For example, each team can have two or more larger goals, two or more smaller goals, two or more smaller goals whose size can be determined by the rules of the game, and/or two or more larger goals whose size can be determined by the rules of the game, and/or two or more goals whose size can be determined by the rules of the game. In embodiments, the playing objective, playing object and/or any aspect of the game can be adjusted according to the rules of the game.

According to another embodiment, offensive participant spectators can directly follow coaching instructions to help move a digital, real, virtual, holographic, and/or other type of goal 8, for example, on the digital back wall, onto the digital back wall, into any position on the digital wall and/or other wall. out of the floor, across the floor, or anywhere on any other wall and/or structure, and/or any 2-D and/or 3-D space and/or location and/or time, space, or any dimension to a position so that the combatant players can score. Coordination can be required to accomplish the planning and/or interaction between the following, but not limited to offensive participant spectators' strength, speed, distance, accuracy, coaching efforts, cheerleading efforts, the decibel level production of participant spectators 4, 7, and participant spectators' computer, computer game, and/or video game skills, performance and/or abilities and/or game energy produced and/or available. The game play energy and efforts of any entity associated with the game can also be used to move and/or detain players, to move the ball, prevent the ball from moving, deflect the movement of the ball, and/or any other action associated with scoring points and/or scoring a goal and/or defending against the scoring of points and/or scoring a goal and/or according to the rules of the game. Defensive players and/or any other entities associated with the game can move a digital, virtual, holographic, real goal defender, and/or other type of defender in front of the goal to prevent scoring and/or according to the rules of the game. Defensive efforts of any entity associated with the game can also be coordinated to optimize strength, speed, and/or ability to block scoring by offensive combatants and/or according to the rules of the game. Offensive players and/or any other entities associated with the game can move a digital, virtual, holographic, real goal defender, and/or other type of offensive weapon, facilitator, and/or other object/entity in front of the goal to assist with scoring and/or according to the rules of the game. Offensive efforts of any entity associated with the game can also be coordinated to optimize strength, speed, and/or ability to assist with scoring and/or according to the rules of the game. Coordination can be between the following, but not limited to defensive participant spectators' strength, speed, distance, accuracy, coaching efforts, cheerleading efforts, the decibel level of participant spectators, and/or participant spectators' computer, computer game, and video game skills and/or abilities of any entity associated with the game. The game play energy and efforts of any entity associated with the game can also be used to move and/or detain players, to move the ball, prevent the ball from moving, deflect the movement of the ball, and/or any action associated with the game, and/or with the rules of the game and/or according to the rules of the game.

In other exemplary embodiments, interference factors, such as a wall and obstacle position, tensile strength, shape, and any other qualities and/or metrics associated with the game can be adjusted and as such can cause a change in the way the playing object 5 plays off of the wall and obstacles. Accordingly, the interference factor can cause unpredictability and/or generate a desired effect in the play of the game, and game play energy can be based on and/or cause the interference factor, either positively or negatively and/or according to the rules of the game.

In embodiments, one type of participant spectator 4, 7 can be involved in games that do not involve their home team. These participant spectators 4, 7 can either be present at the stadium, at home, at their own home field, and/or at some other remote location. Participant spectators not affiliated with the home or visiting team-known in this embodiment as participant spectators of special interest (PSOSI) 7 can apportion their energy only to one or both teams' defense and not to the offense. In embodiments, higher scores can be more valuable to teams in the overall standings of the league and/or organization associated with the game. In embodiments, higher scores from more goals scored, points accumulated and/or the like can be more valuable to teams in the overall standings of the league and/or or in any and all organizations associated with the game. As such, PSOSI 7 playing defense for any team may provide a benefit to their own home team in a game between two teams that are not their home team and/or more than two teams as the overall scores may be lower with the help of PSOSI playing defense.

In embodiments, PSOSI 7 can apportion their actions to one or both teams' defense and/or offense. In embodiments, PSOSI 7 can apportion their actions to a specific player combatant. In embodiments, PSOSI 7 can apportion their actions to a specific play. In embodiments, PSOSI 7 can apportion their actions to defend against and/or help the play of a specific participant spectator 4, 7 and/or group of participant spectators. In embodiments, PSOSI 7 can apportion their actions any way they choose.

In embodiments, PSOSI 7 can apportion their actions to a specific defensive and/or offensive player combatant. In embodiments, PSOSI 7 can apportion their actions to a specific defensive and/or offensive play. In embodiments, PSOSI 7 can apportion their actions to defend against and/or support the play of a specific participant spectator. In embodiments, PSOSI 7 can apportion their actions any way they choose. In embodiments, PSOSI 7 can apportion their game play energy to any entity associated with the game.

In embodiments, any participant spectators 4, 7 can apportion their actions to their teams' defense and/or offense. In embodiments, any participant spectators 4, 7 can apportion their actions to a specific player combatant. In embodiments, any participant spectators 4, 7 can apportion their actions to a specific play. In embodiments, any participant spectators 4, 7 can apportion their actions to defend against and/or support the play of a specific participant spectator 4, 7. In embodiments, any participant spectators 4, 7 can apportion their actions any way they choose. In embodiments, participant spectators 4, 7 can apportion their game play energy to any entity and/or entities associated with the game.

In embodiments, participant spectators 4, 7, instead of generating game play energy through their actions such as exercise, movement, and/or noise can instead generate game play energy through their actions online and/or on a local computer version of the game against players and/or participant spectators of the other team. In embodiments, participant spectators 4, 7 can play another online and/or local computer game against players and/or participant spectators of the other team, teams, and/or any other entities associated with the game, while the game is being played. In embodiments, participant spectators 4, 7 can be playing a game that directly influences the play of the game and/or the configuration of the field of play 1 in coordination with the spectator coaching staff 12, player coaching staff 9, and/or any other entities associated with the game via their actions.

In embodiments, all the actions under the direction of the spectator coaches 12, the player coaches 9, the cheerleaders 13, the participant spectators, and/or any other entities associated with the game can be converted into game play energy that, can be used directly by the offensive player combatant or combatants, defensive player combatant or combatants, participant spectators and/or any entity associated with the game to do among the following, but not limited to move the virtual, digital, real, holographic and/or other types of goals 8; move the real and/or virtual ball, disc, puck or energy capsule and/or other playing object 5; move, raise, or lower a variety of obstacles on the playing field 1 and/or above and/or near the playing field; raise a real, virtual, digital, and/or holographic goal into a place on the playing field and/or above or near the playing field; move obstacles that might interfere with play on and/or onto and/or near the playing field; or move, change, or manipulate a variety of natural-type obstacles such as water from sprinklers to represent rain or wet conditions and wind generating fans to represent wind interference on the playing field, temperature changes, sunlight, glare, darkness, light and/or the like and/or above, on and/or near the playing field; prevent and/or hinder any or all of the above; and/or any type of actions associated with the game and/or affect, alter, impact and/or influence any type of actions associated with the game according to the rules of the game.

In embodiments, all the actions under the direction of the spectator coaches 12, the player coaches 9, the cheerleaders 13, the participant spectators 4, 7, and/or any other entities associated with the game can be converted into game play energy that can be used directly and/or by the player combatant defense and/or offense to do among the following, but not limited to move the virtual, digital, real, holographic goals, and/or other types of goals 8; move the real and/or virtual ball, disc, puck and/or energy capsule and/or other playing object 5; move, raise, or lower a variety of obstacles on the playing field 1; raise a real, virtual, digital, and/or holographic goal into a place on the playing field and/or above or near the playing field; move obstacles that might interfere with play on and/or near the playing field and/or above and/or near the playing field; or move, change, or manipulate a variety of natural-type obstacles such as, but not limited to, water from sprinklers to represent rain or wet conditions and wind generating fans to represent wind interference on the playing field, temperature changes, sunlight, glare, darkness, light and/or the like and/or above, on and/or near the playing field; assist, promote, prevent and/or hinder any or all of the above; and/or assist, promote, prevent and/or hinder any type of actions associated with the game.

In embodiments, some or all of the actions, under the direction of the spectator coaches 12, the player coaches 9, the cheerleaders 13, the participant spectators 4, and/or any entity associated with the game can be converted into audible cheering; used to generate noise; and/or to interfere with the production or generation of noise.

In embodiments, the in-person and/or the remote spectator participants 4, 7 can have access to their spectator coaching staff 12 and/or team for direction as to how to manipulate (i.e., a coaching influence) the field to improve defensive or offensive positioning, scoring opportunities, player alignment, and the like. Coaching influence can be applied through game play energy by the coaching staff, the spectator participants, and/or any other entity associated with the game to influence the outcome of the game.

In embodiments, the in-person and/or the remote spectator participants 4, 7 and/or the spectator coaching staff and/or anyone else associated with the game can use game play energy to access magnetic force and/or electromagnetic factors and or any other forces or factors or the like to move the virtual and/or real playing object and/or objects and/or any entity associated with the game 5 in a certain way. For example, magnetic or electromagnetic factors can be found within or around the following: virtual or real playing object 5; the playing field or parts thereof; the obstacles; the stadium; the maze; the goals; the walls of the playing field; the cheerleaders; the player combatants; and/or any other aspect of the game if the virtual or real playing objects are magnetized and/or the player combatants and/or other entities associated with the game are wearing magnetized shoes, clothing and/or equipment.

In embodiments, game play factors can be applied that helps and/or hinders players from moving freely across, forward and back, above and/or below the field, and/or up and down the field. For example, players combatants 11 can be wearing magnetic or electromagnetic uniforms and/or other restricting devices, and/or a wearable and/or wearables now available and/or to be designed. Magnetic energy and/or other process may be used to pull, push, and/or prevent the movement of players in certain directions, and/or in any direction, hindering, and/or abetting player progress. In embodiments, wearables and/or other devices may be used to affect the movement of players and/or any entity associated with the game and/or to measure, record, transfer and/or transmit energy according to the rules of the game.

In embodiments, game play factors can be applied that may help and/or hinder the playing objects 5, e.g., the ball, disc, puck, and/or energy capsule, from moving freely across, forward and back, and/or up and down the field. For example, playing objects 5, e. g., the ball, disc, puck, and/or energy capsule, and/or the like can be impregnated with or contain magnetic or electromagnetic material, and magnetic energy or some other process may be used to pull, push, and/or prevent the movement of the playing objects 5 in certain directions, hindering or abetting the movement and progress of the playing objects 5 and/or any entity associated with the game.

In embodiments, the participant spectators 4, 7 can be identified with e.g. affiliated with, connected with, associated with, and/or the like with either one of the two teams and/or more teams on the playing field, and/or the participant spectators of special interest PSOSI 7 are affiliated with their own team that is not presently on the field or the participant spectators may be fans of the sport and not affiliated with any team-PFANS. In embodiments, the participant spectators 4, 7 can be identified with one team in the league and may only be allowed to change affiliations at certain times during the season and during the off-season. In embodiments, PFANS can apportion their game play energy to any entity associated with the game. In embodiments, participant spectators can be under contract with a coach, a player, a team and/or the like.

In embodiments, the participant spectators 4, 7 can have different abilities to influence the various variable game parameters based on the rules of the game. In embodiments, players may be rated by a handicapped system (i.e., an exemplary game play factor) that adjusts their game play energy output according to age, skill, experience, equipment, type of exercise, movement, noise production, computer game ability, and/or participation/involvement in other aspects of the game and/or according to some other aspect of the game and/or characteristic. In embodiments, a participant spectator's game energy production and/or game energy use or the game energy production and/or game energy use of any entity associated with the game can be affected by that entities' handicap with and/or without associated wagering.

In embodiments, the production and or use of game energy by any entity associated with the game can affect the wagering odds of the game or of an aspect of the game according to the rules of the game. The game energy generated can be used to affect a wager related to the game (e.g., a bet made at a physical casino, a bet taken over the Internet, and/or the like), according to any, or all of the following: a predetermined formula; a predetermined contract; a formula generated solely on the performance of one, and or more than one of the entities associated with the game; and/or a formula generated on the performance of one in or more than one entities associate with the game based upon previous performance.

In embodiments, for example, the game can be played in outdoor or indoor stadiums, arenas, and/or venues, e.g., constructed indoors either on top of a hockey rink and/or basketball court. For instance, the playing field in indoor stadiums may be the size of a hockey rink, e.g., about 200′×85′. The field may have glass walls and/or acrylic walls, without or without the ability to transmit, amplify and/or reduce the noise, up to 3 or 4 stories high or higher or lower i.e., less than 1 foot, 1 to 100 feet, to more than 100 feet, to ensure that the ball may be reflected or deflected i.e., played off the wall or played over the wall. There may be players on each of the multiple horizontal floors of activity, with each floor approximately 10 to 20 feet higher and/or less than 10 feet to more than and/or less than 20 feet higher than the floor below and possibly not covering the entire surface above the ground level playing floor so there can be movement between the different floors of play. In embodiments, the game can be played while teams and/or by player coaches, spectator coaches, cheerleaders, players, spectators and/or any entity associated with the game are in a the same and/or different location or locations.

In embodiments, the multiple playing floor layers on which players are located can be made of a transparent, translucent, solid, and/or invisible material and/or force and/or the like (i.e., an exemplary game play factor). The layers can be designed to allow for the players 11 to climb onto/up to the upper levels of play or down to the lower layers of play. Players can also be able to climb up and/or to fall off the walls and/or off of layers of the field of play onto lower layers without injuring themselves. The players can be tethered and/or have jet packs and/or with other ways to control horizontal and/or vertical positioning, and/or positioning anywhere and/or positioning in any dimension, time and/or space. The ground floor and/or the playing field can be hardwood, soft, and/or trampoline material and/or other material that allows for a bounce and/or for a rebound. In one embodiment, the ground floor and/or the playing field and/or the other floors and/or walls can be made of any solid, liquid, gaseous material, and/or magnetic/electromagnetic material. In one embodiment the ground floor and other floors and/or walls and/or playing field can be real, digital, and/or virtual. In one embodiment the ground floor and/or other floors and/or walls and/or playing field can be made of animal, vegetable, and/or mineral material. In one embodiment the ground floor and/or other floors and/or walls and/or playing field are static. In one embodiment the ground floor and/or other floors and/or walls and/or playing field are dynamic, moving, and/or flowing. In one embodiment the ground floor and/or other floors and/or playing field and/or walls are static and/or dynamic, moving, and/or flowing.

In embodiments, the players can be equipped with jet packs and/or other sources of energy that may be activated at various times. In embodiments, the players may be equipped with jet packs and/or other sources to assist with movement that may be activated as a result of actions generated by their own teams' participant spectators 4, 7. In embodiments, the players 11 may be equipped with jet packs or other sources of energy generation devices that may be activated as a result of game play energy generated by their opponents' teams'participant spectators 4 in an effort to disrupt that player or those players' ability to succeed. In embodiments, the players can also be equipped with other types of devices and apparatus that assist and/or limit the movement of the players. In embodiments, the jet packs and/or other devices and/or other sources to assist with movement and/or affect movement can be activated and/or controlled by any entity associated with the game, e.g., participant spectators 4, player coaches and/or staff 9, spectator coaches and/or staff 12, cheerleaders 13, judges 16, and/or referees 17.

In embodiments, certain parts of the playing field can be specifically devoted to being gender specific, e.g., just for females and/or just for transgender individuals, to set up competition amongst individual players. Participant spectators 4, 7 may direct their energies toward manipulating that section of the field or any particular section either under the direction of spectator coaches 12, cheerleaders, player combatants, and/or other participants, and/or independently.

In embodiments, the game play energy that any of the entities associated with the game, e. g., participant spectators 4, player coaches and/or staff 9, spectator coaches and/or staff 12, cheerleaders 13, judges 16, and/or referees 17, and/or others can generate may be distributed slowly, may be distributed as it is generated, may be saved and distributed as a bolus, and/or may be generated and/or distributed according to the objectives and/or rules of the game, for impacting or influencing variable game parameters.

In embodiments, the playing field 1 can include walls at the far ends of the playing field and/or around the entire playing field. The wall can be utilized to keep the playing object and/or objects 5 in play. In embodiments, the walls can be constructed of glass, plastic, any other type of material, and/or combinations. In embodiments, the walls can be formed by game play energy, energy and/or forces, for example, magnetic fields and/or electro-magnetic fields. The walls around the entire playing field may be a digital computer screen and/or barriers to keep the ball in play. The walls can be transparent and/or semi-transparent and/or virtual computer screens so that spectators behind the screen can see both the play as it is occurring on the field and/or also see what is being digitally posted on the glass. In one embodiment, the walls, barriers, endlines, sidelines goals, playing field and/or any of the parts of the game and/or parts of the field of play and/or the like, are static, move and/or can be moved as a result of game energy and/or according to the objectives and/or rules of the game.

In embodiments, the playing field 1 can include moveable digital and/or real goals at both ends of the field, digital and/or goals below the playing surface, on the playing surface, and/or above the playing surface and/or digital, virtual and/or real goals at other locations. Each goal can be separately and/or collectively controlled by spectator coaches 12, the player coaches 9, the cheerleaders 13, the participant spectators 4, and/or the player combatants 11, and/or any entity involved in the game.

In embodiments, the computer screen and/or other display devices that are used and/or viewed by any participants and any other entity associated with the game can contain the digital, virtual, and/or real goal, goal line, goal post, scoring objective and/or the like according to the rules of the game that can be manipulated by the offense's participant spectators. In embodiments, the computer screen and/or other display devices can contain the digital, virtual, and/or real goal, goal line, goal post, scoring objective and/or the like according to the rules of the game that can be manipulated by the defense's participant spectators. In embodiments, the computer screen and/or other display devices can contain the digital, virtual, and/or real goal, goal line, goal post, scoring objective and/or the like that can be manipulated by the offense's and/or the defense's participant spectators and/or the PSOSI and/or other entities associated with the game according to the rules of the game 7. In embodiments, the computer screen and/or other display devices can contain the digital, virtual, and/or real goal, goal line, goal post, scoring objective and/or the like that can be manipulated by the offense and the defense participant spectators and the PSOSI according to the rules of the game 7. In embodiments, the computer screen and/or other display devices can contain the digital, real and/or virtual goal, goal line, scoring objective and/or the like and/or a digital, virtual, and/or real defensive blocker and/or blockers according to the rules of the game. In embodiments, the digital, virtual, and/or real goal, goal line, scoring objective and/or the like and/or a digital virtual, and/or real defensive blocker and/or any entity associated with the game can be manipulated and moved by any of the entities associated with the game, e. g., player combatants 11 (offensive player combatants 2, and/or defensive player combatants 3), participant spectators 4, player coaches and/or staff 9, spectator coaches and/or staff 12, cheerleaders 13, judges 16, referees 17, and/or any other entities associated with the game, according to the rules of the game. In embodiments, the digital, virtual, and/or real offensive assistant and/or assistants and/or any entity associated with the game can be manipulated and moved by any of the entities associated with the game, e.g., player combatants 11 (offensive player combatants 2, and/or defensive player combatants 3), participant spectators 4, player coaches and/or staff 9, spectator coaches and/or staff 12, cheerleaders 13, judges 16, referees 17, and/or any other entities associated with the game, according to the rules of the game.

In embodiments, these variable parameters can apply to any sport and/or game. In embodiments, the goals 8 can be actual, virtual, holographic, and/or digital. In embodiments, there can be any number of goals 8 of any size, shape, and/or position. In embodiments, player combatants 11 (offensive player combatants 2, and/or defensive player combatants 3), participant spectators 4, player coaches and/or staff 9, spectator coaches and/or staff 12, cheerleaders 13, judges 16, referees 17, and/or any other entities associated with the game can be actual, virtual, holographic, and/or digital. In embodiments, the goals 8 can be physically, digitally, holographically, and/or virtually and/or the like present on the playing field, in the playing field positioned on the back wall, positioned on a computer screen, and/or positioned in some other place or in some other way. In embodiments, the goals 8 can also be digitally, holographically, virtually, and/or physically placed at a distance from the back wall so that player combatants 11 can play behind the goal, e.g., the way an ice hockey goal has room behind it for players to maneuver, with or without a playing object. The player combatants and any other entity associated with the game can interact with the playing object using bare hands, their feet, their heads, holding gloves, sticks, bats, and/or the like and/or other devices and/or body parts and/or telepathically and/or likewise according to the rules of the game to move, score, defend and/or the like the playing object 5 and/or any other entity associated with the game.

In embodiments, the goal and/or other variable parameters may or may not be fixed in one place. The actual, virtual, holographic, or digital goal 8 can be purposely moved through the input of the participant spectators 4, 7 and/or any other entities associated with the game who may coordinate their individual and collective energies through a spectator coaching staff 12 devoted to maximizing the value and/or usage of energy input provided by the participant spectators into the game plan or strategy. The participant spectator coaching staff and/or any entity associated with the game coordinates with the player coaching staff 9 and/or any entity associated with the game to manipulate the field, e. g., by moving the goal, adjusting the field, and/or positioning the players on the field, and/or the like according to the rules of the game.

In embodiments, the playing objects 5, for example, ball, disc, puck, and/or energy capsule, real, digital, virtual, holographic, and/or any other object, can be put into play at the beginning of play, after a goal is scored, after certain types of fouls in several different ways, after timeouts, and/or at any other time during the game. For instance, the playing object 5 can be put into play by first physically passing the ball through the participant spectators 4, 7 in attendance who work together to get the playing objects 5 to a specific but potentially ever-changing entry to the field of play so that play between the player combatants can begin.

In embodiments, in-house participant spectators can help launch the playing objects 5 directly onto the field of play and/or participant spectators (in-house and remote) may help propel the ball through a maze and/or obstacle course and/or the like onto the field of play. The playing objects 5 can be put in play by traveling through a real maze and/or obstacle course and/or the like located in the stadium or a virtual maze located in the stadium or elsewhere before reaching the player combatants 11. The playing objects 5 can be dropped into the stands and/or onto the field of play or into play and/or the like from the maze and/or from the like. When the playing object and/or objects e.g., a ball is or balls are in the maze and/or the like it can be navigated by the actions of the coaches, cheerleaders, participant spectators, player combatants and/or any entity associated with the game. In an example, the playing objects 5 can be launched into the stands, the maze and/or into the obstacle course and/or the like through a spring-loaded device just the way a pin ball enters the pinball playing field through a spring-loaded trigger. In an example, the traveling of the playing objects 5 through the maze to gain entry to the field of play may be preferred by the visiting team spectators as they may primarily have a remote presence and perhaps be less able to get the ball onto the playing field through the participant spectators that are in the stadium. Each team may have one or more playing objects 5 in the maze and one or more playing objects, e.g., balls in the stands.

In embodiments, the team can choose their preferred method of their playing objects 5 potential entry onto the field of play. When one of the team's playing objects 5 enters the playing field, the other team's and/or teams' playing object(s) 5 may or may not still be active. In embodiments, there may be a limit on the time advantage one team can have for their playing objects 5 to be in play while the other team's playing object(s) 5 is still in the stands in the maze, and/or the like. Once that time advantage has been achieved, the other team's playing objects 5 may automatically be brought through the maze and/or the stands onto the playing field and put into play. The playing objects 5 can be launched or brought onto the playing field to a position predetermined by the coaches to optimize the advantage to the player combatants. The game can be any digital interactive spectator sport, game or activity in which spectators, players and or any entity associated with the game can participate that increases physical activity, increases community, and/or increases socialization among spectators, players and/or any entity associated with the game.

In embodiments, some measure of home court advantage, disadvantage, and/or no advantage may be determined by the decibel level of participant spectator and/or any other entity associated with the game cheering and/or noise making at their home field and/or venue and/or the like. Cheering, encouragement, noise and the like has been shown to help players and actors in venues perform better and/or worse and therefore participant spectators and/or player combatants and/or any entity associated with the game may perform better and/or worse as a result of noise generated. That in-house participant spectator decibel level and/or any entity associated with the game could be compared directly to the decibel level production per spectator of visiting team cheering and/or visiting team noisemaking and/or by some other measure to give an advantage or disadvantage and/or no advantage or disadvantage to those with more vocal spectators overall, more vocal spectator participants in-house or a greater number of participant spectators in-house and/or remote and/or according to some other formula.

In embodiments, the noise delivered in the stadium can be directly related to the noise generated in the stadium. In embodiments, the noise delivered into the stadium can be directly and/or indirectly related to the noise generated remotely according to the rules of the game. In embodiments, the noise delivered to remote locations can be directly and/or indirectly related to the noise generated in the stadium and/or in other locations according to the rules of the game. In embodiments, the noise delivered into the stadium can be directly related to a combination of the noise generated in the stadium and generated remotely. In embodiments, the noise delivered into the stadium can directly result in energy given to and/or taken from one or more the teams and/or other entities associated with the game. In embodiments, the chanting, singing, music and/or noise delivered into the stadium and/or generated in the stadium and/or elsewhere can be converted to game energy and/or can be directly related to a specific effect on the players, an impact, influence and/or effect on the game, on any entity associated with the game and/or on the outcome of the game, i.e., the chanting, music, and/or singing related to the desire of the participant spectators and/or other entities associated with the game for a specific outcome may make it more likely that a specific outcome, influence and/or effect may occur. Cheering and songs sung together by fans and/or music played may enhance fan identity, may enhance a sense of belonging, may decrease social isolation and/or encourage participant spectators to exercise more and/or make more noise and/or sing more and/or create more game energy.

Further, cheerleaders 13 can be present around the stadium to lead the spectators in song, music, noise-making and cheer, encouraging teamwork, support, and/or encourage more exercise, singing, noise making and the like among the spectators and/or other entities associated with the game. Cheerleaders 13 can receive energy from participant spectators and or other entities associated with the game that they can use to move themselves around the field, to move more quickly around the field, and/or to jump higher, further, longer, and/or the like in an effort to get their participant spectators to be more involved and/or to make more noise, cheering, music, to sing specific chants, songs, and/or cheers, to generate more energy, and/or to win at computer games and/or to generate more game energy.

Participant spectators 4, 7 in the stadium can choose for their noisemaker machines 18 to generate energy and/or noise. Remote participant spectators can determine how to apportion their energy and noise. They can choose for their noisemaking, their noisemaker machines, and/or their cheering to generate energy to be used to affect the outcome of the game and/or to influence the game on the playing field or to generate noise in the stadium, and/or to generate a combination of energy and noise in any combination they desire and/or to be used in any way they desire.

Cheerleaders 13 can be involved in leading in-house and/or remote participant spectators 4, 7 in song, cheers, to generate noise and/or to make music. In another embodiment, some measure of advantage, disadvantage, and/or no advantage may be determined by the decibel level of noise generated by participant spectators 4, 7 and/or any other entity associated with the game at the visiting field, home field or venue and/or the like. In another embodiment, some measure of advantage, disadvantage, and/or no advantage may be determined by the decibel level of noise generated by participant spectators 4, 7 and/or any other entity associated with the game at remote locations and/or the like.

Cheerleaders 13 either real or virtual can be involved in encouraging in-house and/or remote participant spectators 4, 7 to start/continue generating game energy (e.g., at a faster rate) by way of some physical activity. In one embodiment, some measure of home court advantage, disadvantage, and/or no advantage may be determined by the level of energy generated directly from exercising by participant spectators 4, 7 and/or any other entity associated with the game at a home field/venue, a visiting field/venue, a remote location, and/or the like. In another embodiment, cheerleader(s) 13 may have routed, to a specific participant spectator 4, 7, personalized content encouraging that participant spectator to start/continue exercising, singing chanting, and/or making noise to generate game energy.

In embodiments, some participant spectators 4,7 may or may not be exercising and/or playing digital games, but their game energy to help their team may be solely, partially, and/or fully determined by their loudness, clarity of vocal participation, the production of music and/or involvement with the team cheers. Energy generation devices 6 can be utilized for energy generation as a result of the efforts of cheerleaders' 13 moves, enthusiasm, teamwork, and/or the successful encouragement of the participant fans. The energy can be released for use as it is generated and/or saved for later use. Cheering, encouragement, noise and the like has been shown to help players and actors in venues perform better and therefore participant spectators and player combatants may perform better as a result of noise generated. Remote participant spectators can choose for their noisemaking, their noisemaker machines, and/or their cheering to generate energy, to generate noise in the stadium, and/or to generate a combination of energy and noise in any combination they desire.

In embodiments, gambling, gambling odds, digital currency, and/or other types of financial activities can be incorporated into certain aspects of the play, including, but not limited to buying game play energy, betting on the outcome of plays, betting on the performance of player combatants, enhancing and/or worsening the performance of any entity associated with the game e.g., participant spectators (including themselves), and/or the outcome of the game and/or buying methods and/or equipment to enhance or detract from the performance according to the rules of the game. In embodiments, game play energy from exercise, cheering, noise making and/or the like can affect gambling, digital currency, and other types of financial activities that can be incorporated into certain aspects of the play, including, but not limited to buying game play energy, betting on the outcome of plays, betting on the performance of player combatants, enhancing and/or worsening the performance of any entity associated with the game e.g., participant spectators (including themselves), and/or the outcome of the game and/or buying methods and/or equipment to enhance or detract from the performance according to the rules of the game. In embodiments, participant spectators, PSOSI, cheerleaders, player combatants and coaches, and/or other entities may or may not bet against the team to which they are affiliated, themselves, and/or aspects their own teams' play. In embodiments, referees and judges may or may not be able to bet on any team in the league during their tenure as an approved referee and/or judge depending on schedules, rules, etc.

In embodiments, judges can be incorporated into certain aspects of the play that require independent third-party judgment for the activities. For example, judges can be used to determine when certain parts of the electromagnetic and/or magnetic floor and/or forces and/or the like, goals, and/or obstacles may be activated so that all and/or some of those on the playing field may be prevented and/or detained from moving and/or have their movement enhanced while all those at elevation may or may not have their movements be detained, prevented and/or enhanced. For safety reasons and/or according to the rules of the game, a warning might be given to player combatants to prevent injuries that might occur were they to be electromagnetically suddenly stopped from moving without warning were the electromagnetic charge on the field to be activated without warning and/or where they to be suddenly moved without warning. Participant spectators, cheerleaders, coaches, and/or any entity associated with the game may all be given advanced notification of this type of magnetic stoppage of play when the electromagnetic charge on the field is to be activated to help with their strategy and to help the player combatants avoid injury and develop a strategy around the time that play will be stopped, started and/or resumed. Participant spectators, cheerleaders, coaches, and/or any entity associated with the game may all be able to determine when certain parts of the electromagnetic or magnetic floor, goals, obstacles and/or other variable parameter of the game may be activated so that all or some of those on the playing field and/or some of the playing objects may be prevented or detained from moving or have their movement enhanced while all those at elevation above the field of play may or may not be detained from moving and/or have their movement enhanced. In embodiments, judges can be incorporated into any and all aspects of play that may or may not require independent third-party judgment.

While the entities above are described as having certain roles in the game and/or any game, the entities can switch roles, share roles, and/or have multiple roles. For example, in embodiments, player combatants, participant spectators, coaches, cheerleaders, referees, judges, and/or anyone else associated with the game can exchange roles, responsibilities, energy generating ability and/or benefits. In embodiments, the player combatants, participant spectators, coaches, cheerleaders, referees, judges, and/or anyone else associated with the game can assume one or more roles, responsibilities, energy generating ability, and/or benefits of the game.

In embodiments, the game can apply to any game. In embodiments, game energy can be used for any game activity including e-sports. In embodiments, the entities associated with the game can be participating in and/or associated with an e-sport event. The entities playing esports can fully or partially gain tokens, weapons, points, victory, energy and/or other benefits and/or the like and/or penalties and/or the like according to the rules of the game by their own exercising, cheering, noise making, music making and/or the playing computer games, and/or playing virtual games, board games, video games, on-line games and/or fantasy games and/or the like and/or from energy generated through their teammates who are exercising, cheering, noise making, music making, and/or the playing of computer games and/or playing virtual games, board games, video games, on-line games and/or fantasy games and/or the like. In embodiments, players playing esports can fully or partially gain tokens, weapons, points, victory, energy and/or other benefits and/or the like and/or penalties and/or the like according to the rules of the game from participant spectators and/or other entities associated with the game who are exercising, cheering, noisemaking and/or playing a game on a computer, and/or playing virtual games, board games, video games, on-line games and/or fantasy games and/or the like.

In embodiments, the entities associated with a game can be participating in an e-sport, and/or associated with any Olympic sport and/or e-sport event such as archery, baseball, chess, cycling, dance, motor sport, rowing sailing, and/or taekwondo and/or any other type of event, whether virtual, real-world, and combination thereof. For example, in embodiments, individuals and/or teams of players on spin cycles, stationary bicycles and/or other exercise equipment or the like and/or exercising and/or cheering and/or noise making and/or playing on a computer, can combine the individual, individuals, team, and/or teams game energy generated to compete against the other individuals and/or teams in any type of board game, real games, sports competition, virtual games and/or fantasy games and the like and/or any individual and/or group activity, and/or any individual and/or group athletic activity and/or competition where spectator involvement is desirable and/or able to be applied and/or where game energy could be added to the way a game and/or competition and/or the like is played and/or scored. In embodiments, gambling, gambling odds, digital currency, and/or other types of financial activities can be incorporated into certain aspects of the play of esports and/or any game, including, but not limited to buying game play energy, betting on the outcome of plays, betting on the performance of player combatants, enhancing and/or worsening the performance of any entity associated with the game e.g., participant spectators (including themselves), and/or the outcome of the game and/or buying methods and/or equipment to enhance or detract from the performance according to the rules of the game.

In embodiments, one or more exercise equipment and devices for providing spectator data can provide both aerobic exercise, anaerobic exercise, and/or muscle-strengthening exercise. For example, an entity associated with the game can generate energy by pedaling a stationary bicycle, forward and/or backward. Additionally, the stationary bicycle can generate energy through the movement of the handlebars. For example, the handlebars can swivel clockwise and counterclockwise, move up and down, move side to side, and/or move forward and backward. Additionally, for example, the handlebars can provide resistance during movement for muscle strengthening through hydraulics, resistance, weight stacks, magnetic and/or electromagnetic resistance, springs, levers, pulleys, bands and/or the like, in any and all directions. All components of the stationary bicycle including, but not limited to, the wheels, handlebars, peddles, crank shaft, seat, seat post and stem, frame, hub and spokes, and/or the like can be free to move and/or have resistance applied to the movement in any and all directions through hydraulics, resistance, weight stacks, magnetic and/or electromagnetic resistance, springs, levers, pulleys, bands and/or the like. As such, the stationary bicycle can provide aerobic exercise, anaerobic exercise and/or muscle-strengthening exercises. The direction of pedaling and the direction of the handlebars can be converted into specific game energy and/or movement in the present time and dimension and/or anywhere in time and/or space. The stationary bicycle can also be equipped with a controller to specifically control the use of game energy. In embodiments, exercise equipment e.g., treadmills, elliptical trainers, climbers, rowing machines and/or the like, can generate energy that can be used for any purpose according to the rules of the game and/or exercise equipment and/or the like can generate energy that specifically converts to forward and/or backward movement, side to side movement, clockwise and/or counterclockwise movement. The exercise equipment and/or the like can also be equipped with a controller to specifically control the use of game energy.

In one embodiment, each of the following muscle strengthening exercises would generate the same amount of game energy according to an example game energy calculation: lifting 200 pounds one time; lifting 100 pounds twice; lifting 2 pounds 100 times; lifting 1 lb. 200 times; and lifting ½ pound 400 times. In another embodiment, a distance each of these weights are lifted would factor into the conversion of exercise activity to game energy. In another embodiment, a time taken to lift this amount of weight would factor into the conversion of exercise activity to game energy. In another embodiment, an age of the participant spectator or any other entity associated with the game performing the exercise would be part of the conversion of exercise activity to game energy. In another embodiment, the previous performance and/or performances of the participant spectator or other entity associated with the game performing the exercise activity would factor into determining the absolute value of the conversion of exercise activity to game energy. In another embodiment, the rules of the game would be factored into the conversion of exercise activity to game energy. In another embodiment, any or all of the above factors and/or the like could be involved in determining the value of the game energy generated from lifting a weight, and or any other type of muscle strengthening, anaerobic or aerobic exercise.

In one embodiment, any of the following exercise activities would create game energy: running a marathon; running 1 mile; running 10 km; walking for one hour; walking for 15 minutes; riding a bicycle on a road bike; riding a mountain bicycle on an obstacle course; riding a spin bike during an exercise class; jumping rope for 10 minutes; jumping rope with a weighted rope; climbing stairs; performing calisthenics; etc. The amount of game energy generated by each of these activities could be determined by any or all of the following factors: the calories generated; the time taken to perform the exercise; the age of the person performing the exercise; the previous performances of the person performing the exercise; the role in the game of the person performing the exercise; the role of the recipient receiving the game energy; the recipient of the game energy; the events occurring during the game by other spectators, and or by any other entities associated with the game; whether the game energy is released in an ongoing way, or as a bolus of energy and or the like; and/or the like.

In embodiments, entities associated with the game, e.g., players and/or spectators can use exercise equipment and/or the like in a variety of locations on which they can register, score and/or store energy points i.e., provide spectator data. Exercise equipment can be found in gyms, fitness stores, and/or at any location. In embodiments, to promote cheering and noise making, the approved exercise equipment and noisemaking equipment that can generate game energy can be located within and/or outside of soundproof booths. The exercise equipment, noise-making equipment, and/or online games, virtual games, and/or board games, can be proprietary or non-proprietary.

While the game and/or any and/or all aspects of the game is described above in examples including one or more teams competing to score goals, the game can include any type of activity and any number of teams and/or any number of entities associated with the game. While the game is described above in examples including one or more teams competing to score goals, the game can include any type of activity and any number of teams associated with any number of other entities who can create game energy that can be used to influence, impact and/or affect the game and/or the outcome of the game. In embodiments, individual players, teams of players and/or spectators can compete against each other by using the game energy they generate using exercise equipment and/or cheering and/or noise making and/or playing on a computer in a tug-of-war game and/or in any competition and/or esport. In embodiments, individual players, teams of players, and/or spectators can compete against each other in real games, virtual games, board games, video games, on-line games and/or fantasy games and the like, by using the game energy they generate using exercise equipment and/or cheering and/or noise making and/or playing on a computer and/or with their minds and/or bodies. In embodiments, individual players, teams of players, spectators and/or any entity associated with the game can compete against each other in real games, virtual games, board games, video games, on-line games and/or fantasy games and/or the like, by using the game energy spectators and/or any entity associated with the game generate using exercise equipment and/or cheering and/or noise making and/or playing on a computer. In embodiments, individual players, teams, spectators and/or any entity associated with the game can compete against each other in real games, virtual games, board games, video games, on-line games and/or fantasy games and the like, by using the game energy spectators and/or any entity associated with the game generate using exercise equipment and/or cheering and/or noise making and/or playing on a computer. In embodiments, spectators and/or any entity associated with the game can generate and store energy before games start, while games are paused, while games are played, during timeouts, and/or during halftime, etc.

In embodiments, the participant spectators and/or entities associated with the game can alter the playing field associated with the game. In embodiments, the participant spectators and/or entities associated with the game can alter the environment associated with the game. In embodiments, the participant spectators and/or entities associated with the game can alter the odds associated with the game. For example, the energy generated can actually “tilt the ice,” e.g., the energy could change the playing field so that instead of being horizontal, it could be tilted in the direction that makes it more likely that the outcome of the competition favors the more energy-generating team, e.g., the team having more game energy.

In embodiments, the amount of participation time an entity devotes to the game can influence the game. For example, spectators, players, coaches, other entities and/or teams associated with the game who devote more active time as a participant spectator or as another entity associated with the game and/or are able to meet some other determinant of involvement related to their association with the game will be positively and/or negatively handicapped and/or not handicapped positively and/or negatively in one way or another. For example, a higher participation time can cause the game energy generated to be increased proportional to the participation time. In another embodiment the amount of time that one is permitted to spend producing energy per week and/or during a certain period of time can and/or will not be limited according to the rules of the game.

In embodiments, entities and/or teams associated with the game, e.g., spectators and other entities associated with the game, can generate energy that can be used in online games or any type of virtual event and/or any type of fantasy event and/or any type of board game and/or any type of fantasy game and/or any individual activity and/or group athletic activity and/or competition and/or game where spectator involvement is desirable and/or able to be applied and/or where game energy could be added to the way the game is played and/or scored and/or influenced.

In embodiments, the energy generated during the game and/or over a period of time can be used for playing of the game and/or for the playing of different games. In embodiments, entities associated with the game, e.g., spectators, can generate energy to be used in a variety of games. In embodiments, energy dissipates according to the rules of the game and therefore must be used in a timely fashion. In embodiments, energy may or may not dissipate overtime and can be stored for a finite period of time and/or for an infinite period of time.

In embodiments, for example, the game being played can be a soccer game, whether real-world, virtual, and/or combinations thereof. For example, 22 participant spectators can be assigned to each real, virtual, fantasy, board and/or digital soccer game. Each participant spectator, assigned to be an assistant, adjunct, aide, designee, auxiliary and/or the like to one of the players in the match, can, use the energy they generate through exercising, cheering, noise making, playing an online game and/or the like, to influence the game and/or the player and/or players to whom they are assigned and/or the player's teammates to function optimally and/or to prevent the player, players. team and/or the like from functioning optimally. In embodiments, the participant spectators can be assigned a role and/or select a specific role, such as goalie and/or assist a goalie and/or influence a goalie. In this example, if a participant spectator picks a specific role such as goalie, the game will commence once all the roles are filled by players. Additionally, a group of spectators may form a team of spectators for a game and/or play against another group of spectators and/or play against a previously established team of participant spectators and/or against a team created by the game. Additionally, one or more teams of spectators and/or participant player and spectators can form a league to play games against one another. In one embodiment, a group of people can choose and/or be chosen to be player combatants and/or a group of people can choose and/or be chosen to be participant.

In embodiments, any entities and/or teams associated with the game can be static and not change roles, change roles at any and/or at all times according to the rules of the game, change roles during a game, change roles between a game, and/or the like. In embodiments, any number of spectators, entities associated with the game and/or teams can be assigned to fill the roster of a sporting event with each spectator, entities associated with the game and/or teams assigned to one or more of the players, and/or entities associated with the game using the energy they generate through exercising, cheering, noise making or playing a sport, an online game in any type of virtual event and/or any type of fantasy event and/or any type of board game and/or any type of fantasy game and/or in any other way they choose and/or are able to generate game energy and/or contribute to the play of the game, their own play, the one or more players to whom they are assigned, any of the players on the field, and/or the play of their teammates. In embodiments, any number of spectators and/or entities associated with the game can be assigned to fill the roster of an online game or any type of virtual event and/or any type of fantasy event and/or any type of board game and/or any type of fantasy game with each participant assigned to be one or more of the players or to provide game energy to one or more of the players, using the energy they generate through exercising, cheering, noise making and/or playing an online game. In embodiments, participant spectators, coaches, cheerleaders, referees, judges and/or anyone else associated with the game can exchange roles, responsibilities, energy generating ability and/or benefits. In embodiments, the players, the spectators and/or others involved in this game and/or other games can be in the same and/or separate location or locations, with a team and/or teams at the same and/or different locations competing against each other and/or working in concert with each other. In embodiments, the player combatants, the participant spectators and/or others involved in a sports action game and/or other games such as Madden Football and/or the like can be in the same and/or separate location or locations, with teams at the same and/or different locations competing against each other and/or working in concert with each other to affect, influence, impact, and/or enhance the game and/or the like.

In embodiments, entities associated with the game, e.g., player combatants, participant spectators, coaches, cheerleaders, referees, judges and others associated with the game, can have access to team psychologists, hypnotists, relaxation experts, peak performance experts, team psychiatrists, team physicians, yoga instructors, fitness experts and/or others and/or the teaching of and/or from others who can help promote optimal physical health and fitness, emotional health and fitness, optimal wagering and/or optimal game playing performance.

In embodiments, the game can include related competitions, events and games, e.g., fantasy sports, gambling, etc., which can be influenced by the entities associated with the game. For example, just as an entity associated with a game can hinder and/or promote the movement of player combatants with jet packs, magnets, opening/closing goals, etc., when participating in Fantasy Games such as Fantasy Football, the value and/or movement of players can be adjusted according to the amount of exercise, cheering, noisemaking and/or computer game involvement and/or the like that is dedicated to the performance of those Fantasy Football athletes by the spectator participants in the Fantasy Football league. The same can apply to Fantasy Baseball and/or the like. In embodiments, just as bets can be placed on the outcome or on the performance of a specific player or on any performance or portion and/or portions of a game when placing bets and or wagering on the outcome of a sporting event and/or the like, the betting odds (or wage parameter) of a bet can be adjusted according to the amount of exercise, cheering, noisemaking and/or computer game involvement and/or the like according to the rules of the game and/or the rules of the Fantasy Game that is expended and dedicated to the outcome of the bet by that participant spectator and/or any entity associated with the game.

In an embodiment, the subject disclosure provides a wagering system that includes a data communication device for transmitting and/or receiving electronic and/or electromagnetic signals of spectator data; and a processor configured to receive spectator data from a participant spectator of an event via the data communication device, wherein the spectator data is based on a physical activity and/or mental activity of the participant spectator, and updates a wage parameter of the event based on the received spectator data in real time. The event can be any game, as discussed above, or any conventional game, such as football, baseball, basketball, soccer, tennis and/or the like. The wage parameter can be the odds of winning a game, the spread to win a game, the over/under odds, and any other odds currently used on sports betting and/or to be developed.

While an objective of the game can be to score goals and win competitions, the subject disclosure provides systems and methods for promoting physical activity and/or mental activity of spectators of a spectatable event, e.g., as many spectators as possible both in person and in remote locations anywhere in the world participating in an active way that may improve the physical health and conditioning and mental health and well-being of these participant spectators and/or others associated with the game through the actions in which they engage to generate game play energy through activities by exercising, cheering, chanting, music and/or noise and/or other energy generating activity and/or other mental and/or body activity associated with and/or during competition. Involvement as a participant spectator 4, 7 can improve the physical and mental conditioning, the physical and emotional fitness, the physical and mental health, the social connection and/or the sense of belonging of any and/or all the spectators and/or any entity associated with the game who become actively and purposely involved in a cause and a competition, participating while spectating, becoming participant spectators 4, 7. When one observes the present sports world, although the number of in-person and remote spectators can be quantified, the ability of spectators to legally influence outcomes has not been able to be fully harnessed.

The subject disclosure, in which in-person and remote spectators and others engage to generate game play energy, by exercising, cheering, chanting, making music and/or noise and/or the like and/or other energy generating activity and/or other mental and/or body activity associated with and/or during and/or related to competition is a way for in-person, remote spectators and/or others to interact with each other to promote the physical health and conditioning and mental health and well-being and performance of these participant spectators and players and others. Further, the systems and methods as disclosed promote a feeling of belonging, important in a society and in a world that may be becoming more anonymous and in which many people feel isolated. Embodiments of the present disclosure promote multigenerational interaction as any and/or all people of all ages and/or those people with disabilities and/or abilities can all participate. Moreover, embodiments of the present disclosure give individuals of all ages, including pre-teens and teenagers who were felt were not “good enough” to help fill the rosters of a variety of teams and/or clubs and/or the like and/or all others a system and/or method to exercise, a system and/or method to be part of a team and/or a system and/or method to decrease social isolation, increase a sense of belonging and/or feel part of the community as participant spectators and/or as for any other entities associated with the game regardless of ability to engage in a game and/or to engage with others. Their personal attributes and/or other attributes of a person may or may not be used to adjust their contribution to the game.

This invention utilizes and/or builds upon the existing infrastructure of athletic activities and/or other events and/or games yet to be invented to help solve an unmet need for aerobic and muscle-strengthening activities for nearly 80 percent of the population who are not exercising according to the 2018 guidelines, 50% who are not meeting the key guidelines for aerobic physical activity and/or to combat the social isolation and loneliness that adversely affect over 50% of the population. Physical inactivity is the fourth leading cause of global mortality according to WHO, and loneliness and social isolation increase the risk of premature death by 26% and 29%, respectively. This disclosure offers a new way for all of humanity to exercise and got involved as a spectator. Exercise can slow or delay the progression of various medical conditions and diseases such as diabetes, cancer, and the like.

Further, the digital world has become a part of our family lives socially, in business, at work and in medicine, but sports involvement in the digital world and in the digital environment lags. Esports, a competition between video gamers that also provides spectator viewing entertainment is a group of relatively new sports that are passively watched by many while played by few. Embodiments of the present disclosure bring the digital world both to the playing field and to the players as well as to the spectators, the coaches, the cheerleaders, referees, judges, and/or other entities associated with the game.

Embodiments of the present disclosure also bring exercise to the spectators and/or other entities associated with the game and take the energy from the spectators to the playing field, coaches, cheerleaders, and other entities associated with the game, combining the digital world and the fantasy world with the real world of sports for all to enjoy, combining the efforts of player combatants with the efforts of participant spectators to the benefit of both player and spectator by promoting health and involvement by and for all. In embodiments, player combatants 11 (offensive player combatants 2, and/or defensive player combatants 3), participant spectators 4, player coaches and/or staff 9, spectator coaches and/or staff 12, cheerleaders 13, judges 16, referees 17, and/or any entities associated with the game can all benefit from being involved with the game, as aerobic exercising and muscle-strengthening methods and activities promote health for all those involved. Additionally, the use of equipment and/or virtual reality can reduce the physical contact between players, thereby reducing contact injuries.

Generally, it is well-known that being physically active is one of the most important steps that people of all ages can take to improve their health. Flexibility is an important part of physical fitness. Stretching exercises are effective in increasing flexibility, and thereby can allow people to more easily do activities that require greater flexibility. Some benefits of physical activity can be achieved immediately, such as reduced feelings of anxiety, reduced blood pressure, improvements in sleep, some aspects of cognitive function, and insulin sensitivity. Other benefits, such as increased cardiorespiratory fitness, increased muscular strength, decreases in depressive symptoms, and sustained reduction in blood pressure, require a few weeks or months of participation in physical activity. Physical activity can also slow or delay the progression of chronic diseases, such as hypertension and type 2 diabetes. Further, physical activity also helps in reducing obesity and its harmful effects. Benefits persist with continued physical activity.

The instant application allows people of all ages, backgrounds, and abilities to improve their health while actively and/or socially participating in the game. The instant disclosure also meets an unmet need of improving their health while observing the game. Embodiments of the present disclosure allow people of all ages, backgrounds, and abilities to adopt this game and/or games associated with this disclosure as a lifetime sport.

FIG. 2 is a block diagram of an example computer system 200 according to an example of the present disclosure. For example, the computer system 200 may be used to implement any of the processes or be a part or portion of any of the components of FIG. 1, as well as to provide computing resources as described herein e.g., the system for game play influenced by spectator activity described above. In various embodiments, the computer system 200 may include a processor 202, a memory 204, and an input/output (I/O) interface 206. In various embodiments, the processor 202 may be used to implement various functions and features described herein, as well as to perform the method implementations described herein. While the processor 202 is described as performing implementations described herein, any suitable component or combination of components of the computer system 200 or any suitable processor or processors associated with the computer system 200 or any suitable system may perform the steps.

The processor 202 can be and/or include a processor, a microprocessor, a computer processing unit (CPU), a graphics processing unit (GPU), a neural processing unit, a physics processing unit, a digital signal processor, an image signal processor, a synergistic processing element, a field-programmable gate array (FPGA), a sound chip, a multi-core processor, and so forth. As used herein, “processor,” “processing component,” “processing device,” and/or “processing unit” can be used generically to refer to any or all of the aforementioned specific devices, elements, and/or features of the processing device.

The memory 204 can be and/or include a computer processing unit register, a cache memory, a magnetic disk, an optical disk, a solid-state drive, and so forth. The memory device can be configured with random access memory (RAM), read-only memory (ROM), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device.

The non-transitory computer-readable storage medium 208 may be any electronic, magnetic, optical, or other physical storage device that stores executable instructions. For example, the non-transitory computer-readable storage medium 608 may be random access memory (RAM), an electrically-erasable programmable read-only memory (EEPROM), a storage drive, an optical disc, or the like. The non-transitory computer-readable storage medium 208 can be encoded to store executable instructions that cause a processor to perform operations according to examples of the disclosure.

The present disclosure may employ a software stack to enlist the underlying tools, frameworks, and libraries used to build and run example applications of the present disclosure. Such a software stack may include PHP, React, Cassandra, Hadoop, Swift, etc. The software stack may include both frontend and backend technologies including programming languages, web frameworks servers, and operating systems. The frontend may include JavaScript, HTML, CSS, and UI frameworks and libraries. In one example, a MEAN (MongoDB, Express.js, Angular JS, and Node.js) stack may be employed. In another example, a LAMP (Linux, Apache, MySQL, and PHP) stack may be utilized. Any suitable programming language can be used to implement the routines of particular examples including Java, Python, JavaScript, C, C++, assembly language, etc. Different programming techniques can be employed such as procedural or object oriented. The routines may execute on specialized processors.

The specialized processor may include memory to store a set of instructions (also known as computer instructions). The instructions may be either permanently or temporarily stored in the memory or memories of the processing machine. The processor executes the instructions that are stored in the memory or memories in order to process data. The set of instructions may include various instructions that perform a particular task or tasks, such as those tasks described above. Such a set of instructions for performing a particular task may be characterized as a software program.

### DRAWING REFERENCE SYMBOLS AND NUMERALS

1 Playing field with electro-magnetic flooring system and hard, soft, and buoyant surfaces, walls, and landings as well as movable obstacles, hazards, and adjustable conditions of all types
2 Offensive player combatants
3 Defensive player combatants
4 Participant spectators
5 Ball, puck, disk, energy capsules, and other types of playing objects which can be used to score a goal according to the rules of the game and that may contain magnetic or electromagnetic material
6 Energy generation/controlling/distribution devices
7 Participant Spectators of special interest—PSOSI
8 Goals
9 Player coach(es) and coaching staff
10 Energy collection/distribution device
11 Player combatants (equipped with energy collection and distribution devices) and wearing magnetic clothing
12 Spectator coach(es) and coaching staff
13 Cheerleaders
14 Communications Network
15 Server
16 Judges
17 Referees
18 Noise generators/cheering

### ASPECTS OF THE INVENTION

A system is described with a data communication device for processing signals of spectator data characterized by a memory having computer instructions executable by a processor configured to: generate data corresponding to a spectatable event having a variable game parameter, the variable game parameter directing execution of the spectatable event, receive, via the data communication device, the spectator data from a second data communication device of a participant spectator of the spectatable event, wherein the spectator data is based on a physical activity of the participant spectator, and update the variable game parameter of the spectatable event based on the received spectator data. The system above wherein the spectatable event comprises the participant spectator as a player playing a game to win, increase physical activity, increase social connection, or increase sense of a community. The system above wherein the spectatable event comprises at least one team having at least one player playing a game. The system above wherein the variable game parameter comprises rules of the game, constraints of the game, objectives of a game, a position of a goal, number of goals, size of the goal, shape of the goal, a change in an obstacle of the game, a change in game field condition, a movement of an object of the game, a strike zone, a field dimension, a net position, number of players on the field, number of balls or playing objects, coaching activity, playing activity, a game plan, scoring, defending, an offensive game plan, a player attribute, or a defensive game plan. The system above further comprising an exercise device for generating the spectator data and transmitting the spectator data to the data communication device. The system above wherein the exercise device comprises a smart phone, a smart watch, or a wearable device. The system above wherein the processor is further configured to communicate, to the second data communication device, content configured to encourage performance of the physical activity by the participant spectator. The system above wherein the processor is further configured to output to an electronic display spectator data analytics data. The system above wherein the spectator data is a quantifiable measure of the physical, mental, and metaphysical actions or activities of the participant spectator. The system above wherein the processor is further configured to transfer the quantifiable measure to a player playing a game. The system above wherein the processor is further configured to transfer the quantifiable measure to an influencer. The system above wherein the processor is further configured to update a wage parameter of the spectatable event based on the received spectator data. The system wherein the wage parameter comprises betting odds of the spectatable event.

A computer-implemented method characterized by: generating data corresponding to execution of a spectatable event having a variable game parameter; receiving, via a data communication device, spectator data from a second data communication device of a participant spectator of the spectatable event, wherein the spectator data is based on a physical exercise activity of the participant spectator; and updating the variable game parameter of the spectatable event based on the spectator data from the participant spectator. The computer-implemented method above wherein receiving the spectator data further comprises communicating, to the second data communication device, content configured to encourage performance of the physical exercise activity by the participant spectator. The computer-implemented method above wherein the variable game parameter includes rules of the game, constraints of the game, objectives of the game, a position of a goal, number of goals, size of the goal, shape of the goal, a change in an obstacle of the game, a change in game field condition, a movement of an object of the game, a strike zone, a field dimension, a net position, number of players on the field, number of balls or playing objects, coaching activity, playing activity, a game plan, an offensive game plan, a player attribute, or a defensive game plan. The computer-implemented method above wherein the variable game parameter is updated upon reaching a predetermined threshold value. The computer-implemented method above wherein the physical exercise activity is an aerobic exercise or a muscular strengthening exercise. The computer-implemented method above wherein the physical exercise activity comprises noise making, singing, chanting, cheering, or booing. The computer-implemented method above wherein the physical exercise activity is a type of cognitive, mental, metaphysical, physical, or emotional activity.

1. A system with a data communication device (300) for processing signals of spectator data characterized by a memory (204) having computer instructions executable by a processor (202) configured to:
generate data corresponding to a spectatable event (1) having a variable game parameter, the variable game parameter directing execution of the spectatable event,
receive, via the data communication device (300), the spectator data from a second data communication device (300′) of a participant spectator (4, 7) of the spectatable event, wherein the spectator data is based on a physical activity of the participant spectator (4, 7), and
update the variable game parameter of the spectatable event (1) based on the received spectator data.

2. The system of claim 1, wherein the spectatable event comprises the participant spectator as a player playing a game to win, increase physical activity, increase social connection, or increase sense of a community.

3. The system of claim 1, wherein the spectatable event comprises at least one team having at least one player playing a game.

4. The system of claim 1, wherein the variable game parameter comprises rules of the game, constraints of the game, objectives of a game, a position of a goal, number of goals, size of the goal, shape of the goal, a change in an obstacle of the game, a change in game field condition, a movement of an object of the game, a strike zone, a field dimension, a net position, number of players on the field, number of balls or playing objects, coaching activity, playing activity, a game plan, scoring, defending, an offensive game plan, a player attribute, or a defensive game plan.

5. The system of claim 1, further comprising an exercise device for generating the spectator data and transmitting the spectator data to the data communication device.

6. The system of claim 5, wherein the exercise device comprises a smart phone, a smart watch, or a wearable device.

7. The system of claim 1, wherein the processor is further configured to communicate, to the second data communication device, content configured to encourage performance of the physical activity by the participant spectator.

8. The system of claim 1, wherein the processor is further configured to output to an electronic display spectator data analytics data.

9. The system of claim 1, wherein the spectator data is a quantifiable measure of the physical, mental, and metaphysical actions or activities of the participant spectator.

10. The system of claim 9, wherein the processor is further configured to transfer the quantifiable measure to a player playing a game.

11. The system of claim 9, wherein the processor is further configured to transfer the quantifiable measure to an influencer.

12. The system of claim 1, wherein the processor is further configured to update a wage parameter of the spectatable event based on the received spectator data.

13. The system of claim 12, wherein the wage parameter comprises betting odds of the spectatable event.

14. A computer-implemented method characterized by:
generating data corresponding to execution of a spectatable event (1) having a variable game parameter;
receiving, via a data communication device (300), spectator data from a second data communication device (300′) of a participant spectator (4, 7) of the spectatable event, wherein the spectator data is based on a physical exercise activity of the participant spectator; and
updating the variable game parameter of the spectatable event (1) based on the spectator data from the participant spectator (4, 7).

15. The computer-implemented method of claim 14, wherein receiving the spectator data further comprises communicating, to the second data communication device, content configured to encourage performance of the physical exercise activity by the participant spectator.

16. The computer-implemented method of claim 14, wherein the variable game parameter includes rules of the game, constraints of the game, objectives of the game, a position of a goal, number of goals, size of the goal, shape of the goal, a change in an obstacle of the game, a change in game field condition, a movement of an object of the game, a strike zone, a field dimension, a net position, number of players on the field, number of balls or playing objects, coaching activity, playing activity, a game plan, an offensive game plan, a player attribute, or a defensive game plan.

17. The computer-implemented method of claim 14, wherein the variable game parameter is updated upon reaching a predetermined threshold value.

18. The computer-implemented method of claim 14, wherein the physical exercise activity is an aerobic exercise or a muscular strengthening exercise.

19. The computer-implemented method of claim 14, wherein the physical exercise activity comprises noise making, singing, chanting, cheering, or booing.

20. The computer-implemented method of claim 14, wherein the physical exercise activity is a type of cognitive, mental, metaphysical, physical, or emotional activity.
